(19) United States
(12) Patent Application Publication
(10) Pub. No.: US 2024/0002786 A1
(43) Pub. Date: Jan. 4, 2024

(54) SHRINKABLE HYDROGELS

(71) Applicants: THE BRIGHAM AND WOMEN'S HOSPITAL, INC., Boston, MA (US); UNIVERSITEIT UTRECHT HOLDING B.V., Utrecht (NL)

(72) Inventors: Yu Shrike Zhang, Bostn, MA (US); Tina Vermonden, Utrecht (NL); Cari Schuurmans, Utrecht (NL)

(21) Appl. No.: 18/036,924

(22) PCT Filed: Nov. 19, 2021
(86) PCT No.: PCT/US2021/060113

§ 371 Date: May 15, 2023

Related U.S. Application Data
(60) Provisional application No. 63/116,011, filed on Nov. 19, 2020.

(51) Int. Cl. C12N 5/00; B33Y 70/00; B33Y 40/20; B29C 64/30

(52) U.S. Cl. CPC C12N 5/0062; B33Y 70/00; B33Y 40/20; B29C 64/30; C12N 2513/00; C12N 2533/70; C12N 2537/10; B29C 64/124

(57) ABSTRACT

A method of shrinking a hydrogel is described. The method includes contacting a polyionic hydrogel with a polyionic solution including an ionic polymer having a net opposite charge from that of the polyionic hydrogel for an amount of time sufficient to decrease the volume of the polyionic hydrogel. Hydrogel compositions made using this method, and methods of using these hydrogels in 3D printing are also described.

### CONTINUING APPLICATION DATA

This application claims the benefit of U.S. Provisional Application Ser. No. 63/116,011, filed Nov. 19, 2020, the disclosure of which is incorporated by reference herein.

### STATEMENT REGARDING FEDERALLY SPONSORED RESEARCH

This invention was made with government support under Grant Numbers K99CA201603, R00CA201603, R21EB025270, R21EB026175, and R01EB028143, awarded by the National Institutes of Health. The Government has certain rights in this invention.

### BACKGROUND

Three-dimensional (3D) printing has become a collection of enabling biofabrication technologies to generate volumetric structures featuring high complexity through robotically controlled dispensing or stabilization processes. The programmed fabrication further makes the procedures highly reproducible. As such, 3D printing is finding widespread applications in biomedicine including, but not limited to, tissue engineering, microphysiological systems, and biomedical devices. Nevertheless, existing printing strategies all have their minimally producible resolutions, which are factors of multiplexed parameters, such as the printer hardware and ink properties. Miri et al., Lab a Chip 19, 2019-2037 (2019). For example, in extrusion printing using hydrogels as inks, the resolutions are typically sub-millimeter for the dispensed microfibers. The same holds true for microfluidic coaxial printing, where the diameters of the created hollow microfibers usually fall in the range of a couple hundred micrometers or larger. Although some other printing strategies, such as those based on light (e.g., two-photon lithography) can achieve varying degrees of higher resolutions, their instrumentation is usually complicated limiting the broader adoption for general use. Tromayer et al., Sci. Rep. 8, 17273 (2018).

To this end, efforts have conventionally been focused on improving the printer hardware or ink properties. Examples in extrusion printing include using smaller nozzles or using inks of high viscosity values, both of which inevitably elevate the need for much higher forces during the dispensing processes, inducing significantly increased shear stresses on the encapsulated cells. Even though, the resolution improvements are still limited. Meanwhile, the progress in printer hardware or tuning the ink formulations may not always be straightforward, leaving these methods still impractical for some applications.

More recently, an implosion fabrication method was proposed, in which a swollen hydrogel matrix was used to photopattern metals, semiconductors, and biomolecules, followed by acid-driven shrinking to achieve nano-sized structures. Oran et al., Science 362, 1281-1285 (2018). While efficient for two-photon lithography, this method is not amenable to most other 3D printing modalities due to the necessity of a preexisting, swollen hydrogel matrix to allow anchoring points for secondary biomolecules or inorganic species subsequently patterned in the volumetric space. Further, such shrinking is unstable and would revert once the stimulus is removed. Finally, the harsh implosion conditions (i.e., low pH) utilized are largely incompatible with living cell-based applications. Accordingly, there remains a need for improved methods of hydrogel shrinkage for use in 3D printing and other applications.

### SUMMARY

The 3D printing of hydrogels is a rapidly evolving field with the capacity to create complex hydrated structures with widespread applications in the field of biomedicine. An important aspect of 3D printing technologies is the attainable spatial resolution of a printed object. Typically, printing resolution is related to the printer hardware and software as well as the choice of the material. Smaller feature sizes are also achievable by processing the printed objects and can lead to large reductions in post-printing hydrogel volume and dimensions. Our study reports a unique method of resolution enhancement purely relying on post-printing treatment of hydrogel constructs. By immersing a 3D-printed patterned hydrogel consisting of a hydrophilic polyionic polymer network in a solution of polyions of the opposite net charge, shrinking can rapidly occur resulting in various degrees of reduced dimensions comparing to the original pattern. This phenomenon, caused by complex coacervation and water expulsion, enables us to reduce linear dimensions of printed constructs while maintaining cytocompatible conditions in a cell type-dependent manner.

In addition, predictable water expulsion from hydrogels through complexation of polycations into anionic networks is systematically investigated. Using a library of polyelectrolytes, an equation is established that can empirically describe the shrinking capacity of a hydrogel. The two main factors influencing the shrinking capacity are the initial hydrogel macromer concentration and the polymer-network interaction strength. Hydrogel shrinking can further be controlled by tuning the anionic-to-cationic charge ratio absorbed into the network. The gel dehydration rate is tunable through polycation diffusion-related parameters. As an example of the use of this post-processing technique, a volumetrically printed hydrogel could reduce its volume up to 18× without altering the object's shape fidelity or aspect ratio. Furthermore, printed hollow objects were shrinkable without collapse of their internal structures. Importantly, post-processing of volumetrically printed hydrogels has been shown to lead to sub-50 micrometer feature sizes for this technique (specifically, 42±6 μm reported here). Hydrogel shrinking greatly increases 3D hydrogel printing resolution and the descriptive model presented here is possibly broadly useful for hydrogel-based additive manufacturing applications.

### BRIEF DESCRIPTION OF THE FIGURES

FIGS. 1A-1F provide graphs and images relating to the general mechanism of complexation-induced hydrogel shrinking. a Schematics showing the shrinking effect based on charge compensation. b Photograph showing size change of fabricated HAMA hydrogel (1.0 w/v %) before (lower) and after (upper) shrinking in 2.0 w/v % MMw chitosan dissolved in 1.0 v/v % acetic acid aqueous solution. c Schematic representation of a HAMA hydrogel disc together with the dimensions and corresponding quantitative analyses showing the dimensions and volume changes before and after shrinking in 2.0 w/v % MMw chitosan dissolved in 1.0 v/v % acetic acid aqueous solution. d Corresponding quantitative analyses showing the shrinking of HAMA hydrogels in perchloric acid solution (pH=1.0) or in 1.0 v/v % acetic acid aqueous solutions (pH=4.7) with 2.0 w/v % of chitosan of different molecular weights and types. e Confocal images showing the diffusion of FITC-Q. chitosan solution in PBS into a 2.0 w/v % HAMA hydrogel at 3 and 24 h of shrinking. The bright-field image at 0 h serves as the size reference of the initial hydrogel. f Photograph showing size change of HMw chitosan hydrogels (2.0 w/v %), where the lower one was swollen in 1.0 v/v % acetic acid aqueous solution and the upper one was shrunken in 2.0 w/v % alginate in 1.0 v/v % acetic acid aqueous solution. **P<0.01; one-way ANOVA (c, d, compared with the values of corresponding as-prepared samples); mean±s.d, (n=3).

FIGS. 2A-2J provide graphs and images showing the direct extrusion printing of HAMA constructs and their shrinking behaviors. a Schematics showing the concept of shrinking printing, where a printed hydrogel structure is post-treated to reduce its size and achieve higher resolution. b, c Printability mapping of HAMA inks at different concentrations and extrusion pressures. d Photographs (upper) and micrographs (lower) showing size changes of printed HAMA hexagons (2.0 w/v %) immersed in 2.0 w/v % HMw chitosan dissolved in 1.0 v/v % acetic acid aqueous solution during the 24 h shrinking process. e, f Corresponding quantitative analyses of size changes of the printed HAMA hexagons (2.0 w/v %), include e side-to-side distance and f thickness, during the 24 h shrinking process. g Photographs (upper) and micrographs (lower) showing size changes of printed HAMA hexagons (2.0 w/v %) at 2 h and 24 h of shrinking in different concentrations of HMw chitosan (0.5-5.0 w/v %) dissolved in 1.0 v/v % acetic acid aqueous solution, h, i Corresponding quantitative analyses of size changes in h side-to-side distance and i thickness. j Vector-field maps comparing the 24-h shrinking images (magenta) to the corresponding 2-h shrinking images (green) by a B-spline-based non-rigid registration algorithm, where the overlaps appear in white and the grids show local distortions. Note that the 2-h shrinking images have been rescaled to match the sizes of the 24-h shrinking images to enable comparisons. **P<0.01; one-way ANOVA (e, f, compared with the values of corresponding as-prepared samples); mean±s.d. (e, f, n=40; h, i, n=10).

FIGS. 3A-3I provide graphs and images showing the sacrificial printing of microchannel-embedded HAMA constructs and their shrinking behaviors. a Schematic of the sacrificial printing process using an extrusion-printed Pluronic F-127 microfiber as the fugitive ink and subsequent shrinking of the construct. b Photographs showing the size change of the HAMA construct (2.0 w/v %) along with the embedded microchannel before (upper) and after (lower) 24 h of shrinkage in 2.0 w/v % HMw chitosan dissolved in 1.0 v/v % acetic acid aqueous solution. c Corresponding quantitative analysis of diameter change of the microchannel before and after shrinking. d Schematic of the sacrificial printing process using a MEW-printed PCL grid as the fugitive ink and subsequent shrinking of the construct. e-g Photographs (left) and micrographs (right) showing the size change of the HAMA construct (2.0 w/v %) along with the embedded microchannel printed (e), washed (f) and shrunken (g) in 2.0 w/v % HMw chitosan dissolved in 1.0 v/v % acetic acid aqueous solution (24 h). h, i, Corresponding quantitative analyses of changes in h diameter of the microchannel and (i) length of the grid before and after shrinking. **P<0.01; two-tailed Student's t-test (c), one-way ANOVA (h, i, compared with the corresponding as-printed structures); mean±sd (n=40).

FIGS. 4A-4G provide graphs and images showing the coaxial printing of cannular HAMA-based constructs and their shrinking behaviors. a Schematic illustrations of the core-sheath coaxial nozzle used as the printhead, where the ink is delivered through the sheath flow and the CaCl₂ solution is co-delivered through the core flow. b Printing of the cannular construct and its subsequent shrinkage. c, d Photographs and micrographs showing the size changes of the tubes, coaxial-printed with inks containing different concentrations of HAMA (0.5-2.5 w/v %), before and after 24 h of shrinkage in 2.0 w/v % HMw chitosan dissolved in 1.0 v/v % acetic acid aqueous solution. e, f, g Corresponding quantitative analyses of diameter (e, inner diameter; f, outer diameter; g, wall thickness) changes before and after shrinkage. **P<0.01; one-way ANOVA (e, f, g, compared with the corresponding as-printed structures); mean±s.d. (n=40).

FIGS. 5A-5E provide graphs and images showing the biocompatibility of the shrinking process and shrinking in the presence of cells. a Schematics showing the single shrinkage (upper) and the successive shrinkage (lower). b Micrographs showing live (green)/dead (red) staining of MCF-7 cells embedded in GelMA/HAMA constructs following a single shrinking process (upper) and a successive shrinking process (lower). c Corresponding quantitative analyses of the numbers of live/dead cells during the two type of shrinking processes. d Fluorescence micrographs of MCF-7 cells in GelMA/HAMA constructs without any treatment (control) as well as after successive shrinkage and single shrinkage, stained for Ki67 (red), F-actin (green), and nuclei (blue). e Corresponding quantitative analyses of the percentages of Ki67+ stained nuclei in the three groups. **P<0.01; one-way ANOVA (compared with the control group on Day 1), mean±s.d. (b, c n=10; d, e n=1, deviations obtained from eight distinct layers of a confocal stack for each sample).

FIG. 6 provides schematic representations of the water expulsion technique used to post-process printed hydrogels. To improve the spatial resolution of anionic hydrogel structures, the hydrogel is incubated over time in an aqueous solution containing polycations to induce water expulsion and subsequent shrinking. Upon introduction of the hydrogel network to the polycations, electrostatic interactions between the negatively charged matrix and positively charged polymers will be favored over the present counterion interactions, causing expulsion of counterions. Due to the neutralization of the charges, the hydrogel will become more hydrophobic, causing water to be expulsed from the network.

FIGS. 7A-7F provide graphs showing A: Volumes of GelMA A, gelMA B, or HAMA hydrogels of varying macromer wt % after incubation in a 2.0 wt % pDADMAC solution (M_{w}: 400-500 kDa). B: volumes of HAMA hydrogels upon incubation in solutions containing pDMAEMA (pH 5.5, M_{w}: 35 kDa), Q. chitosan (M_{w}: 50-100 kDa), or pDADMAC (M_{w}: <100 kDa C: Volumes of HAMA hydrogels upon incubation in solutions of pDMAEMA of different pH but identical ionic strengths (pDMAEMA M_{w}: 35 kDa). D: Volumes of HAMA hydrogels upon incubation with pDADMAC with an average M_{w} of <100, 200-350, and 400-500 kDa. E: Volumes of 1.0 wt % HAMA hydrogels upon incubation with solutions of pDADMAC of different molecular weights as a function of the ionic strength of the buffer solutions. F: Percentage of shrinking (with maximum attainable shrinking at 100%) of 1.0 wt % HAMA hydrogels shrunken with varying amounts of polycations. All measurements were done after full shrinking (room temperature, pH 7.4, 170 mM of ionic strength) using 2.0 wt % polycation solutions (allowing the polycation to be present in excess) unless indicated otherwise. For GelMA A hydrogels with a macromer concentration of <3.0 wt %, gelMA A (DM: 100%) with a M_{w} of 50-100 instead of 40-50 kDa was used. For HAMA hydrogels with a macromer concentration of <2.5 wt %, HAMA with a M_{w} of 1530 kDa was used, for higher macromer concentrations HAMA with M_{w} of 70 kDa was used. The Vᵢ was 56 mm³. Solid lines in 2ABCD represent the fit of Eq. 1 through the observed data points, where the dashed lines indicate extrapolation of Eq. 1. The shown points are averages+/−SD of triplicates.

FIGS. 8A-8F provide graphs showing A: Hydrogel volume over time for a 1.0 wt % HAMA hydrogel incubated in a pDADMAC (M_{w}: 200-350 kDa) solution of various concentrations. Solid lines represent the fit of Eq. 4 to the data. B: Hydrogel shrinking kinetic factor k (as calculated via Eq. 4) interpolated from the data shown in FIG. 8A. Solid line represents a linear fit to the data points. C: Hydrogel shrinking factor k for 1.0 wt % HAMA hydrogels incubated at various temperatures. Solid line represents an exponential fit. D: Hydrogel shrinking factor k for 1.0 wt % HAMA hydrogel discs of varying volumes but similar aspect ratios. Solid line represents an exponential fit. E: Times until full HAMA hydrogel shrinking when incubated with pDADMAC of varying M_{w}. F: Volume for 8.0 wt % HAMA hydrogels incubated with pDADMAC of varying M_{w} and with buffer only. Note that E and F share the same legend. All experiments were done with 1.0 wt % HAMA hydrogels (Vᵢ=56 mm³) at room temperature, pH 7.4, 170 mM of ionic strength using 2.0 wt % pDADMAC (M_{w}: 200-350 kDa) solutions unless stated otherwise. All data points represent the average+/−SD of n=3, measured at various time points; error bars are only visible when larger than the data point symbols and symbolize experimental error in 3A and F, and the 95% confidence interval of the determined rate constant k in 3BCD.

FIGS. 9A-9E provide graphs and images showing A: A volumetrically printed perforated hydrogel disc made from 1.0 wt % HAMA before and after shrinking for 24 h in a 2 wt % pDADMAC solution (room temperature, pH 7.4, ionic strength: 17 mM, polymer M_{w}: 400-500 kDa). B: Quantified spoke widths of the printed object shown in FIG. 9A before and after shrinking. C: Estimated volumes based on calibrated microscopy measurements of the printed object shown in FIG. 9A before and after shrinking. D: A volumetrically printed hydrogel gyroid made from 4.0 wt % gelMA B before and after shrinking for 24 h in a 2 wt % pDADMAC solution (room temperature, pH 7.4, ionic strength: 17 mM, polymer M_{w}: 400-500 kDa). Visualized using micro-computed tomography (PCT). E: Quantified spoke widths of the printed objects shown in FIG. 4D before and after shrinking. F: Estimated volumes of the printed objects shown in FIG. 4D before and after shrinking.

FIGS. 10A-10F provide graphs and images showing A: Volumetrically printed hydrogels (1.0 wt % HAMA) prior to shrinking. B: Volumetrically printed hydrogels (1.0 wt % HAMA) after shrinking through incubation with varying amounts of pDADMAC, with 100% defined as the minimum amount of pDADMAC required to fully shrink the printed construct (pH 7.4, 170 mM of ionic strength, average Mw of pDADMAC: 400-500 kDa). C: Dimensional shrinking ratios (construct heights pre-shrinking/post-shrinking) as a function of the amount of added pDADMAC. D: Volume shrinking ratios (construct volumes pre-shrinking/post-shrinking) as a function of the amount of added pDADMAC. E: Zoomed-in μCT images of original and fully shrunken volumetrically printed stars (1, 2 are original and 3, 4 are fully shrunken). F: The smallest feature sizes of the volumetrically printed stars (points are indicated in FIG. 10E), before and after shrinking; n=5

FIGS. 11A & 11B provide chemical structures of the anionic macromers and polycations used; A: All anionic macromers used in this study and their respective places on a bar indicating their negative charge density. 1: HAMA, 2: gelMA A, 3: gelMA B. B: All cationic polymers used to shrink anionic hydrogels in this work, placed on a bar indicating their respective positive charge densities. 4: pDMAEMA-PEG, 5: Q.Chitosan, 6: Lysozyme, 7: pDMAEMA, 8: pDADMAC. Note that both polyanions and polycations depicted are drawn in their charged state without their respective counterions.

FIG. 12 provides a graph showing the shrinking ratio (Vi/Vs) of hydrogels made with 1.0, 1.5, or 2.5 wt % macromer concentration as a function of average macromer negative charge density, incubated in 2 mL of 2.0 wt % pDADMAC (Mw: 200-350 kDa, added in excess) in 170 mM of ionic strength and pH 7.4 PBS at room temperature for 12 h. Hydrogels were composed of gelMA B combined with HAMA to change their average charge density. Specifically, hydrogels contained 100, 75, 50, 25, or 0% of HAMA.

FIG. 13 provides a graph showing shrunken 1.0 wt % HAMA hydrogel volumes after incubation in an 85-mM ionic strength, pH 7.4 PBS solution at room temperature supplemented with 2.0 wt % lysozyme, Q.Chitosan, or pDADMAC of different DMs. Initial hydrogel volume was 56 mm³. HAMA macromer initial Mw was ˜70 kDa and DM was 12, 24, or 34%. N=3±SD

FIG. 14 provides a graph showing shrunken 1.0 wt % HAMA hydrogel volumes after incubation in an 85-mM ionic strength, pH 7.4 PBS solution at room temperature supplemented with 2.0 wt % lysozyme, Q.Chitosan, or pDADMAC of different molecular weights. Initial hydrogel volume was 56 mm3. HAMA macromer initial Mw was ˜70, 289, or 1530 kDa and the DM was 25%.

FIG. 15 provides a graph showing shrunk 4.0 wt % GelMA B hydrogel volumes after incubation in an 85-mM ionic strength, pH 7.4 PBS solution at room temperature supplemented with 2.0 wt % lysozyme, Q.Chitosan, or pDADMAC of different DMs. Initial hydrogel volume was 56 mm3, and DM was 30, 60, or 100%.

FIG. 16 provides a graph showing shrunk 4.0 wt % GelMA A hydrogel volumes after incubation in an 85-mM ionic strength, pH 7.4 PBS solution at room temperature supplemented with 2.0 wt % lysozyme, Q.Chitosan, or pDADMAC of different DMs. Initial hydrogel volume was 56 mm3, and DM was 30, 60, or 100%.

FIGS. 17A & 17B provide graphs showing A: HAMA hydrogels (initial volume: 56 mm3) shrunken with pDMAEMA with an average Mw of 11, 19, or 35 kDa. These hydrogels were shrunken with 2.0 mL, 2.0 wt % pDMAEMA (added in excess) in 170 mM of ionic strength, pH 5.5 at room temperature for 12 h. B: HAMA hydrogels shrunken with pDMAEMA-PEG with an average Mw of 10+5, 20+5, or 39+5. These hydrogels were shrunken with 2.0 mL, 2.0 wt % pDMAEMA-PEG (added in excess) in 170 mM of ionic strength, pH 5.5 at room temperature for 12 h.

FIG. 18 provides plotted graphs of Eq. 1 for a hypothetical hydrogel of macromer concentration P, Vᵢ of 100 mm³, and a factor of 2, 5, or 10.

FIG. 19 provides a graph showing the times until full hydrogel shrinking for 1.0 wt % HAMA hydrogel discs of varying volumes but similar aspect ratios. Solid line represents an exponential fit. Shrinking was done at room temperature, pH 7.4 and 170 mM of ionic strength using 2.0 wt % pDADMAC (200-350 kDa) polycation solutions of increasing volume

FIGS. 20A-20F provide graphs and images showing A: Effects of several additives (0.1 wt % photoinitiators added) on the shrinking of 1.0 wt % HAMA hydrogels incubated with 2.0 wt % pDADMAC (200-350 kDa), 170 mM of ionic strength, and pH 7.4. B: DLP-printed hydrogel design (left) and printed and shrunken (right). C: The same gel printed and shrunken, from a macroscopic perspective. D-F: The hydrogel design, fabricated hydrogel, and shrunken hydrogel of a volumetrically printed 0.5 wt % HAMA hydrogel shrunken with 2.0 wt % pDADMAC (400-500 kDa), 17 mM of ionic strength, pH 7.4. Scale bar is 500 micrometers for SI11CEF

### DETAILED DESCRIPTION

A method of shrinking a hydrogel is provided. The method includes contacting a polyionic hydrogel with a polyionic solution including an ionic polymer having a net opposite charge from that of the polyionic hydrogel for an amount of time sufficient to decrease the volume of the polyionic hydrogel. Hydrogel compositions made using this method, and methods of using these hydrogels in 3D printing are also provided.

### Definitions

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4 This applies regardless of the breadth of the range.

As used herein, the term “about” means ±10% of the recited value.

A weight percent (wt. %) of a component, unless specifically stated to the contrary, is based on the total weight of the formulation or composition in which the component is included.

Polyionic, as used herein, refers to a compound that is a multiply charged ion, such as a polycation or polyanion. Because the polycations or polyanions are typically part of a polymer or protein, that include significantly more charged ions (i.e., 10 or more, 50 or more, or 100 or more) than a mere plurality of ions.

### Methods of Shrinking a Hydrogel

The ability to shrink a hydrogel, and in particular a hydrogel ink, can be useful for improving the resolution that can be achieved using hydrogels, particularly in 3D printing. In one aspect, the present invention provides a method of shrinking a hydrogel that includes contacting a polyionic hydrogel with a polyionic solution including an ionic polymer having an opposite charge from that of the polyionic hydrogel for an amount of time sufficient to decrease the volume of the polyionic hydrogel.

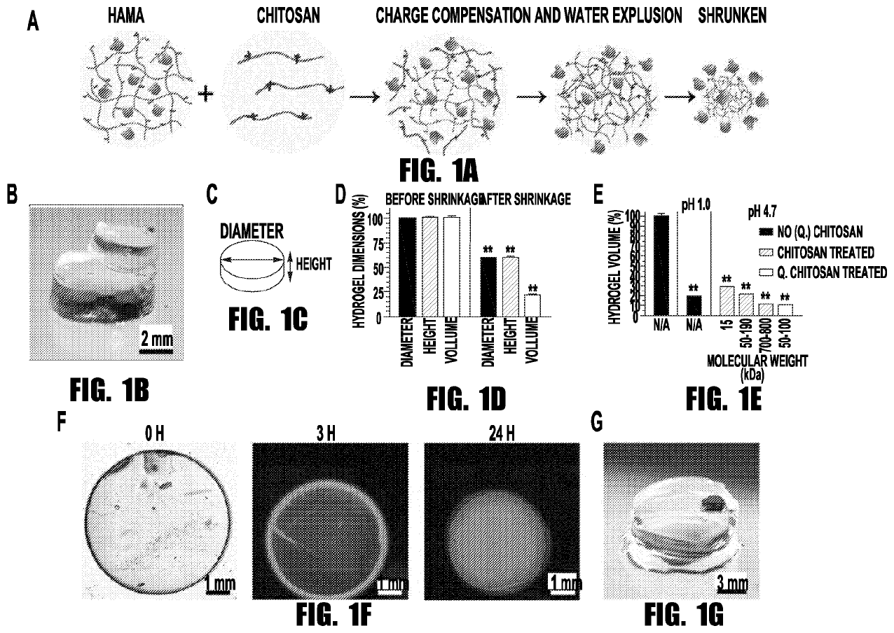

The inventors have demonstrated that when a polyionic hydrogel is contacted with a polyionic solution including an ionic polymer having an opposite charge that this results in an electrostatic interaction, leading to counterion release which causes the hydrogel to lose water and shrink in size. The mechanism behind hydrogel shrinkage is shown in FIGS. 1A and 6. In some embodiments, the hydrogel is a polyanionic hydrogel and the polyionic solution is a polycationic solution, while in other embodiments the hydrogel is a polycationic hydrogel and the polyionic solution is a polyanionic solution.

Contacting, as used herein, refers to placing the polyionic hydrogel and the polyionic solution in physical contact so that they can interact with each other. In some embodiments, contacting involves placing the polyionic hydrogel into the polyionic solution.

A shrunken hydrogel is one that has a decreased volume compared with that of the polyionic hydrogel before being contacted for a sufficient amount of time with the polyionic solution. Preferably, the shrunken hydrogel also retains the same geometry it had before shrinking, but with reduced dimensions. The decrease in the volume, also referred to as the hydrogel shrinking capacity, can vary depending on a variety of factors including the initial hydrogel macromer concentration, the chemical nature of the specific hydrogel (e.g., the charge density), the buffer pH of the polyionic solution, and the electrostatic interaction between the polycation and the anions present in the mixture. In various embodiments, the method can result in a 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, or even 20 fold decrease in the volume of the hydrophilic polyionic hydrogel, with the fold value representing the ratio of the original volume to the final volume.

The method requires that the hydrophilic polyionic hydrogel be contacted with a polyionic solution for an amount of time sufficient to decrease the volume of the polyionic hydrogel. The amount of time required can vary depending on a variety of factors, such as the temperature and the nature of the polyionic hydrogel and the polyionic solution. The inventors have determined that the shrinking rate is directly related to the penetration of the polyionic compound from the polyionic solution into the polyionic hydrogel. In some embodiments, the shrinking of the hydrogel is complete within 12 hours. In some embodiments, the shrinking of the hydrogel is complete within 18 hours. In further embodiments, the shrinking of the hydrogel is complete within 24 hours. In yet further embodiments, the shrinking of the hydrogel is complete within 36 hours.

The method includes the step of contacting a polyionic hydrogel with a polyionic solution. A hydrogel may be defined as a three-dimensional, hydrophilic or amphiphilic polymeric network capable of taking up large quantities of water. The networks are composed of homopolymers or copolymers, are insoluble due to the presence of covalent chemical or physical (ionic, hydrophobic interactions, entanglements) crosslinks. The crosslinks provide the network structure and physical integrity. In some embodiments, the hydrogels are hydrophilic hydrogels, meaning they have an affinity for water.

The hydrogel is prepared by crosslinking suitable macromers to form the hydrogel. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. The hydrogel should be electrostatically charged, or modified to bear electrostatically charged groups. Examples of hydrogels based on crosslinked synthetic polymers include but are not limited to electrostatically charged versions of (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. See A. S Hoffman, Adv. Drug Del. Rev 43, 3-12 (2002). In some embodiments, the hydrogel is an alginate or polyacrylamide hydrogel.

The hydrogels used herein are also polyionic. Polyionic hydrogels are hydrogels bearing multiple charged ions. The multiple charged ions can have either an overall positive or a negative charge, resulting in polyionic hydrogels that are either polycationic or polyanionic. Polycationic or polyanionic hydrogels can be obtained by using polymers modified to include carbohydrates such as hyaluronic acid or chitosan that bear carboxyl, sulfate, or phosphate groups to provide a negative charge, or amino groups to provide a positive charge. In some embodiments, DNA or other nucleic acid based materials having a negative charge due to the presence of phosphate groups can be used.

The polyionic hydrogel is prepared by crosslinking hydrogel macromer in an aqueous (e.g., water) solution. Suitable cross-linking agents for preparing the polyionic hydrogels described herein are known to those skilled in the art. For example, alginate macromers can be cross-linked using CaCl₂ solution. The method of shrinking the hydrogel can be carried out on a polyionic hydrogel that has been prepared earlier, or the method can further include the step of preparing the polyionic hydrogel.

The inventors have determined that the initial macromer concentration can have a significant effect on the hydrogel shrinking capacity. Macromer concentrations ranging from 0.1 to 10 weight percent (relative to the aqueous solution) can be used. In some embodiments, the macromer concentration is prepared from hydrogel macromers having a weight percentage of 10% or less, 5% or less, 2.5% or less, 1% or less, or 0.5% or less in an aqueous solution. In further embodiments, the macromer concentration can range from 0.1% to 5%. 0.1% to 2.5%, 0.1% to 1%, or 0.5% to 1% by weight.

In some embodiments, the polyionic hydrogel is a polyanionic hydrogel. Examples of polyanionic hydrogels include hydrogels including negatively charged carbohydrates such as xanthan, hyaluronic acid, or alginate. Other examples of polyanionic hydrogels include hydrogels including negatively charged polypeptides, such as gelatins. Specific examples of polyanionic hydrogels include hydrogels selected from the group consisting of hyaluronic acid methacrylate, gelatin methacryloyl (type A and type B), and alginate.

In some embodiments, the polyionic hydrogel is a polycationic hydrogel. Examples of polycationic hydrogels include hydrogels including positively charged carbohydrates, proteins, peptides, or synthetic polycations. Specific examples include chitosan and lysozyme.

The method also includes the use of a polyionic solution including an ionic polymer (e.g., polymer or protein) having a net opposite charge from that of the polyionic hydrogel. Ionic polymers include polypeptides (and proteins made up from polypeptides). Contacting the hydrogel with the polyionic solution causes the hydrogel to shrink based on the mechanism described herein. In some embodiments, where the hydrogel is a polyanionic hydrogel, the polyionic solution is a polycationic solution, while in other embodiments where the hydrogel is a polycationic hydrogel, the polyionic solution is a polyanionic solution. The compound of the polyionic solution must be water soluble, and capable of entering into the hydrogel where is results in a loss of water and attendant shrinkage of the hydrogel.

In some embodiments, the ionic polymer of the polyionic solution is polycationic ionic polymer that provides a polycationic solution when added to an aqueous solution. Examples of polycationic compounds are those including polyamines. In some embodiments, the ionic polymer is selected from the group consisting of chitosan, quaternized chitosan, lysozyme, poly [2(dimethylamino)ethylmethacrylate], poly [2(dimethylamino)ethylmethacrylate]-co-polyethylene glycol, and polydiallyldimethylammonium chloride solutions.

In some embodiments, the ionic polymer of the polyionic solution is polyanionic ionic polymer that provides a polyanionic solution when added to an aqueous solution. Examples of polyanionic compounds are those including polycarboxylates, polysulfates, or polyphosphates. In some embodiments, the ionic polymer is alginate or hyaluronic acid.

One application of the shrinkable hydrogel is its use in 3D printing, where the shrinking can be used to increase the level of detail/resolution in the product being made. Accordingly, in some embodiments, the method of shrinking the hydrogel further comprises applying the polyionic hydrogel to a surface using a 3D printing method before contacting the polyionic hydrogel with a polyionic solution.

In some embodiments, shrinkage of the polyionic hydrogel is carried out in cell culture medium. Standard cell culture techniques are typically used. In embodiments in which the shrunken hydrogel composition comprises cells and is 3D printed into a printed article, subsequent addition of cells is not required but also not restricted. In such embodiments, a portion of or the entire printed article can be placed under standard cell culture conditions (e.g., temperature, pressure, nutrient concentrations, etc.).

### Shrunken Hydrogel Compositions

Another aspect of the invention provides a shrunken hydrogel composition. The shrunken hydrogel composition includes water and a polyionic polymer, prepared by contacting a polyionic hydrogel with a polyionic solution including a ionic polymer having a net opposite charge from that of the polyionic hydrogel for an amount of time sufficient to decrease the volume of the polyionic hydrogel. In some embodiments, the polyionic hydrogel is a hydrophilic polyionic hydrogel.

The polyionic hydrogel and polyionic solution used to prepare the shrunken hydrogel composition can be any of the hydrogels and solutions described herein. In some embodiments, the hydrogel is a polyanionic hydrogel, and the polyionic solution is a polycationic solution, while in other embodiments, the hydrogel is a polycationic hydrogel and the polyionic solution is a polyanionic solution.

Likewise, any of the specific polyionic hydrogels and polyionic solutions can be used in the preparation of the shrunken hydrogel composition. For example, in some embodiments, the hydrogel is selected from the group consisting of hyaluronic acid methacrylate, gelatin methacryloyl, and alginate.

The method can result in a 2- to 20-fold decrease in the volume of the hydrophilic polyionic hydrogel, with the fold value representing the ratio of the original volume to the final volume. This can be useful to provide structures having features with a smaller size than could be normally obtained, e.g., through 3D printing. In some embodiments, the shrunken hydrogel composition can have features as small as 70 μm, 60 μm, 50 μm, 40 μm, m, or even as small as 20 μm.

In some embodiments, the shrunken hydrogel composition comprises one or more embedded viable cells and/or cell types. In some embodiments, the printed 3D article is a scaffold for depositing and/or growing cellular tissue. A scaffold for cellular growth can have any suitable three-dimensional shape or dimensions. As a non-limiting example, a scaffold can comprise a stack of alternating layers of strands comprising the shrunken hydrogel composition. When the hydrogel contains cells, the cells may be substantially uniformly distributed throughout the hydrogel, or they may be suspended within a part of the hydrogel.

In some embodiments, the shrunken hydrogel composition comprises from about 1×10¹ to about 1×10⁹ viable cells, or from about 1×10² to about 1×10⁸ viable cells, or from about 1×10³ to about 1×10⁷ viable cells, or from about 1×10⁴ to about 1×10⁷ viable cells, or from about 1×10⁵ to about 1×10⁷ viable cells.

Viable cells include prokaryotic and eukaryotic cells. Non-limiting examples of eukaryotic cells include mammalian cells (e.g., stem cells, progenitor cells and differentiated cells). Stem cells have the ability to replicate through numerous population doublings (e.g., at least 60-80), in some cases essentially indefinitely, and also have the ability to differentiate into multiple cell types (e.g., pluripotent or multipotent). Other viable cells include immortalized cells that do not undergo normal replicative senescence, and can proliferate essentially indefinitely. Other living cells include embryonic stem cells, amniotic fluid stem cells, cartilage cells, bone cells, muscle cells, skin cells, pancreatic cells, kidney cells, nerve cells, liver cells, and the like. Viable cells are living cells.

The shrunken hydrogel composition can comprise a viable cell in an encapsulated form. Encapsulated cells are cells or small clusters of cells or tissue that are surrounded by a selective membrane laminate that allows passage of oxygen and other required metabolites, releases certain cell secretions (e.g., insulin), but limits the transport of the larger agents of the host's immune system to prevent immune rejection. Encapsulation can be useful for implanting and/or injecting cells or tissues containing living xenogeneic or allogeneic cells while reducing the risk of immune rejection in a host. This can be useful in treating diseases due to inadequate or loss or secretory cell function, or ailments that would benefit from the addition of certain secretory cells such as acute liver failure, type I diabetes, chronic pain, Parkinson's disease, and other diseases. Other uses of encapsulated cells include, but are not limited to, single cell analysis, high throughput drug screening, and stem cell differentiation at the single cell level. The cells can be encapsulated in a microcapsule of from 50 or 100 micrometers to 1 or 2 mm in diameter. The microcapsules can include one or more living cells, preferably 1 to 10 living cells, more preferably 1 to 5 living cells.

The shrunken hydrogel composition can also include one or more additives. Non-limiting exemplary additives for the ink compositions include diluent synthetic polymers (e.g., PEG, polypropylene glycol, poly(vinyl alcohol), poly(methacrylic acid)), drugs (e.g., antibiotics such as penicillin and streptomycin), cell nutrients (e.g., proteins, peptides, amino acids, vitamins, carbohydrates (e.g., starches, celluloses, glycogen), and minerals (e.g., calcium, magnesium, iron), synthetic or naturally occurring nucleic acids, surfactants, plasticizers, salts (e.g., sodium chloride, potassium chloride, phosphate salts, acetate salts), living cells, and cell components (e.g., elastin, fibrin, proteoglycans). The composition can comprise one or more additives in an amount of 0 wt % to about 25 wt % of the composition, based on total weight of the composition.

### 3D Printing Methods

Another aspect of the invention provides a method of 3D printing. The method includes the steps of applying a composition comprising a polyionic ink in a pattern to form a structure; crosslinking the polyionic ink to provide a polyionic hydrogel; and contacting the polyionic hydrogel with a polyionic solution including a ionic polymer having a net opposite charge from that of the polyionic hydrogel for an amount of time sufficient to decrease the volume of the polyionic hydrogel.

The polyionic ink can be any of the polyionic hydrogels described herein. In some embodiments, the polyionic ink is a hydrophilic polyionic ink. Likewise, the polyionic solution can be any of the polyionic solutions described herein. In some embodiments, the hydrogel is a polyanionic hydrogel, and the polyionic solution is a polycationic solution, while in other embodiments, the hydrogel is a polycationic hydrogel and the polyionic solution is a polyanionic solution.

In some embodiments, the polyionic ink is a stable hydrogel that does not need to be chemically crosslinked. In this case, the method includes the steps of applying a composition comprising a polyionic ink in a pattern to form a structure; and contacting the polyionic ink with a polyionic solution including a ionic polymer having a net opposite charge from that of the polyionic ink for an amount of time sufficient to decrease the volume of the polyionic ink.

3D printing refers to an additive deposition process for constructing three-dimensional single or multi-layered structures disposed on a substrate without using lithography. The features of the structures are defined by computer-aided design/computer-aided manufacturing (CAD/CAM) software. Devices for printing 3D articles are known, such as those disclosed in US Patent Application Publications US20180021140A1 and US20170217091A1, which are each incorporated by reference herein in their entireties.

The shrinkable hydrogels and methods of shrinking a hydrogel disclosed herein can be used in the methods of printing a 3D article. The polyionic hydrogels are useful as “inks” for three-dimensional (3D) printing (also known as direct-write printing) in a variety of different 3D printing methods. In some embodiments, the polyionic ink is applied by being extruded using an extrusion print-head. In other embodiments, the polyionic ink is applied using a stereolithographic printing apparatus. In yet other embodiments, the polyionic ink is applied using a digital light processing printing apparatus. In further embodiments, the hydrophilic polyionic ink is applied using vat-polymerization printing apparatus, such as a volumetric printing apparatus.

The following provides a detailed description of a method of 3D printing utilizing an extrusion print-head. However, while provided in the context of extrusion printing, many of the features described are also applicable to other printing methods.

The substrate can be any suitable base material (e.g., flexible film base, glass plate, metal foil, and the like), and can comprise one or more layers. The surface of substrate can have a temperature which is about the same as the temperature of the polyionic hydrogel, or can alternatively be higher or lower to induce changes in efficiency of the crosslinking reaction.

The polyionic hydrogel is extruded from a deposition nozzle moving relative to the substrate. The movement facilitates formation of single and/or complex shapes of the resultant printed article. The construction of the 3D layered structures is computer controlled and can be done in a laminar fashion. In one configuration, the deposition nozzle (e.g., of the printer) moves in the z direction and the substrate supporting the extruded printing polyionic hydrogel moves in the x and y directions. In another configuration, the deposition nozzle moves in 3 dimensions and the substrate is stationary. In another configuration, the substrate moves in 3 dimensions and the deposition nozzle is stationary. The deposition nozzle can dispense continuously to generate microstrands of the printing composition, or discontinuously to dispense microdrops of the polyionic hydrogel. Liquid flow of the printing composition can be controlled by air pressure (pneumatic nozzle) or using a stepper motor (volume-driven injection), or by other known methods. The strand thickness of the exuded polyionic hydrogel can be modulated by varying the deposition speed, tip diameter, and/or the applied pressure. Strand thickness or the line width can also be a function of viscosity of the printing composition and interaction of hydrogel with the substrate.

The shrinkable hydrogel can be deposited onto the substrate, results in a simple or complex shape or structure disposed on the substrate. 3D printing facilitates the formation of virtually limitless shapes and structures, which can be used to mold structures used to mimic in vivo cell growth environments. In some embodiments, the one or more layers are deposited in a predetermined pattern. Resultant shapes/structures can be separated from the underlying substrate as a free-standing shape or structure if desired. Optionally, the extrusion and deposition steps can be repeated one or more times, applying the same or a different printing composition, each time in contact with or separate from (e.g., for later joining with) a previously deposited layer. Thus, complex structures comprising one or more layers, or one or more printing compositions, can be deposited on the substrate.

In some embodiments, any one or more steps of the 3D printing method can be performed at a temperature from about 1° C. to about 99° C., or from about 10° C. to about 75° C., or from about 20° C. to about 50° C., or from about 25° C. to about 37° C. In some embodiments, all steps of the 3D printing method can be performed at a substantially constant temperature (e.g., no temperature change is required).

The present invention is illustrated by the following example. It is to be understood that the particular example, materials, amounts, and procedures are to be interpreted broadly in accordance with the scope and spirit of the invention as set forth herein.

### EXAMPLES

### Example 1: Complexation-Induced Resolution Enhancement of 3D-Printed Hydrogel Constructs

Here, a strategy of complexation-induced resolution enhancement in 3D printing is described that we term as shrinking printing, through post-treatment of the printed structures, for resolution improvement without requiring changing any of the printer hardware or much of the ink compositions. In particular, we select inks that are anionic, such as those based on commonly used hyaluronic acid methacrylate (HAMA) (Highley et al., Adv. Mater. 27, 5075-5079 (2015)), gelatin methacryloyl (GelMA) (Ying et al., Bio-Des. Manuf. 1, 215-224 (2018)), and alginate (Lee, K. Y. & Mooney, D. J., Prog. Polym. Sci. 37, 106-126 (2012)). Following standard printing procedures, we subject the HAMA-, GelMA-, or alginate-based hydrogel constructs to immersion in a polycationic chitosan solution. Through charge complexation and subsequent expulsion of water from the gels, these printed constructs are found to reduce in their linear dimensions in various degrees.

We conduct our proof-of-concept studies with several 3D printing techniques, including direct extrusion printing, sacrificial printing, and microfluidic hollow fiber printing, where successful shrinking is observed in all cases. Additionally, we prove the wide applicability of our method by demonstrating shrinking of printed polycationic chitosan-based hydrogel constructs, with polyanionic alginate. We finally demonstrate that selected types of cells embedded in the printed hydrogel matrices remain viable upon successive shrinking in contrast to a single, longer shrinking procedure, further revealing the potential applications of our shrinking printing method in the presence of living cells.

Results

Shrinking Behaviors of Polyionic Hydrogels

The carboxyl groups present in HAMA render the hydrogel negatively charged under physiological conditions. When a HAMA hydrogel is placed in the presence of polycations of high charge densities, such as chitosan, a type of glucosamine featuring reasonable biocompatibility and densely cationic nature due to the abundantly available amino groups, charge compensation occurs leading to expulsion of water and eventual size reduction of the HAMA hydrogel (FIG. 1a). Indeed, this phenomenon was previously observed for microgels. Li et al., Biomacromolecules 11, 1754-1762 (2010). We also recently demonstrated, that when a solution of positively charged lysozyme was introduced to microfluidics-fabricated HAMA microspheres, the size of these microspheres was decreased due to a mechanism akin to complex coacervation, facilitating their use in drug delivery. Schuurmans et al., Soft Mater. 14, 6327-6341 (2018).

To prove the concept, we first fabricated cylindrical HAMA hydrogels (1.0 w/v %, D=6 mm, H=2 mm, ˜56.5 μL in volume). When immersed in 2.0 w/v % chitosan (Mw: 50-190 kDa) dissolved in 1.0 v/v % acetic acid aqueous solution for 24 h, the hydrogels shrank to about 60% in height and diameter, leading to a reduction to 21% in volume as compared to the original constructs (FIG. 1b and FIG. 1c). The changes of concentrations of HAMA hydrogels before and after complete shrinkage in chitosan (Mw: 700-800 kDa) were subsequently evaluated. Our calculations revealed that the concentrations increased from the initial 0.5-2.5 w/v % to 7.9-12.1 w/v % after shrinking. Scanning electron microscopy (SEM) images of the HAMA structures before and after shrinking in chitosan (Mw: 700-800 kDa) were obtained. Although the concentrations of HAMA in the shrunken hydrogels were much higher than those of the initial constructs, water was still the main constituent maintaining their hydrogel nature for various relevant applications.

Considering that the molecular weight of chitosan might affect the shrinkage extent, we compared chitosan of different molecular weights (low-molecular weight, LMw: 15 kDa; medium-molecular weight, MMw: 50-190 kDa; high-molecular weight, HMw: 700-800 kDa). All chitosan types had a similar degree of deacetylation (85%). As shown in FIG. 1d, the HAMA hydrogels (1.0 w/v %) at pH=1.0 shrank to 19.0±0.2% of their original size, while the ones in 2.0 w/v % LMw chitosan, MMw chitosan, and HMw chitosan in 1.0 v/v % acetic acid aqueous solutions (pH=4.7) shrank to 28.9±1.1, 21.6±0.9, and 11.0±0.7%, respectively. In addition to the molecular weight, the charge density of the shrinking agent might be an important factor affecting the shrinkage degree. Therefore, we compared three types of chitosan with different degrees of deacetylation (72.5%, 77.8%, and 94.6%) but similar molecular weights (50-250 kDa). The HAMA hydrogels (1.0 w/v %) in 1.0 v/v % acetic acid aqueous solution (pH=4.7) shrank to 55.1±1.5% of their original size, and the ones in 2.0 w/v % chitosan in 1.0 v/v % acetic acid aqueous solutions shrank to 23.5±1.0, 18.5±0.8, and 12.6±0.7% for chitosan with different degrees of deacetylation (72.5%, 77.8%, and 94.6%), respectively. Moreover, the shrinkage ratio with quaternary ammonium salt chitosan (Q. chitosan, M_{w}: 50-100 kDa, 90% of deacetylation degree, 95% of quaternization degree) was observed to be the highest among all chitosan types, at 9.7±0.4% (FIG. 1d).

These results indicated that both the average polymer chain length and the average charge per monomer of the chosen cationic polymer, i.e., chitosan, influenced the extent of shrinkage of the HAMA hydrogels. The strength of electrostatic complexation between two oppositely charged polymers is known to be dependent on the chain length and charge density (i.e., amount of monomers/charges in a single polymer chain). de Kruif et al., Curr. Opin. Colloid Interface Sci. 9, 340-349 (2004). These observations coupled to the fact that shrinking with polycations resulted in lower-volume (i.e., more heavily dehydrated) hydrogels than when compared to a similar hydrogel incubated in a buffered pH 1.0 solution bring us to the conclusion that, the hydrogel shrinks due to the formation of a complex coacervate-like structure between the oppositely charged hydrophilic polymer network and the free polymer. Dehydration is commonly seen in both complex coacervation and precipitation in an electrostatic strength-dependent manner. When the deacetylation degree and thus the average positive charge per monomer remained constant (i.e., 85% of deacetylation degree, +1 per 271.8 Da of charge density), the higher molecular weight of chitosan was, the higher degree of shrinking was observed. The average charge density of every chitosan monomer would also be of great importance; the higher the charge density (+1 per 328.8 Da, +1 per 298.7 Da, +1 per 238.3 Da, for different degrees of deacetylation [72.5%, 77.8%, 94.6%] of chitosan, respectively), the higher the shrinkage ratio.

Moreover, we made two types of bulk HAMA hydrogels (2.0 w/v %) of similar aspect ratios to study effects of the initial hydrogel volume on hydrogel shrinking (cylinders, D=6 mm, H=2 mm, ˜56.5 μL, or D=8 mm, H=3.4 mm, ˜170.9 μL). The thicker structures took longer to shrink, but shrank to a similar degree as the thinner structures did. To investigate the shrinking process, we visualized the polycation diffusion using fluorescein isothiocyanate-labeled Q. chitosan (FITC-Q. chitosan), by incubating 2.0 w/v % HAMA cylinders in a 2.0 w/v % FITC-Q. chitosan solution, and after 3 and 24 h, observations were made with confocal microscopy (FIG. 1e). The results revealed that FITC-Q. chitosan diffused into the entire structure and complexed with the polyanion (HAMA) already at 3 h, yet with most of the FITC-Q. chitosan still located at the periphery. However, after complete shrinking at 24 h, we observed a homogeneous distribution of FITC-Q. chitosan throughout the cylinder, indicating that the shrinking agent fully penetrated the hydrogel matrix, and that the relatively low weight percentage of HAMA used for forming the hydrogel did not hamper the diffusion of large polymer chains of chitosan into the matrix. Subsequent release of FITC-Q. chitosan from the fully shrunken hydrogels was quantified in phosphate-buffered saline (PBS) and followed for 21 days. Only in the first few days, release of FITC-Q. chitosan was observed, which can be attributed to the loss of FITC-Q. chitosan loosely bound (non-ionically) to the surfaces of the hydrogels that was washed off rapidly during the incubation. After the first few days, no further release was observed. The hydrogel volumes did not change over time, indicating that the shrunken constructs remained stable without further significant release of chitosan.

Since alginate is one of the most commonly used hydrogels in 3D printing, we investigated whether our shrinking process was also compatible with alginate hydrogels (2.0 w/v %). After physical crosslinking in a CaCl₂ solution (0.3 M), the alginate cylinders (D=6 mm, H=2 mm, ˜56.5 μL) were obtained and washed in deionized water, or shrunken in 2.0 w/v % of chitosan of different molecular weights and types. The alginate hydrogels in water swelled to 105.9±2.5%, and when incubated in 1.0 v/v % acetic acid aqueous solution, the cylinders shrank to 51.2±1.4% of their original volume, whereas the ones in LMw chitosan, MMw chitosan, and HMw chitosan solutions shrank to 37.6±1.2%, 28.9±1.1%, and 27.7±1.0%, respectively. Moreover, the shrinkage degree in Q. chitosan was validated at 25.4±3.9%, similar to that shrunken by the HMw chitosan. These results indicated that alginate, as a negatively charged polymer similar to HAMA, could also shrink in a polycationic solution using our complexation-based technique.

To investigate whether this approach was generally applicable to polycationic inks as well, we prepared chitosan-based cylindrical hydrogels for further study. HMw chitosan hydrogel (2.0 w/v %) cylinders (D=6 mm, H=2 mm, ˜56.5 μL) crosslinked with different concentrations of glutaraldehyde were incubated either in 1.0 v/v % acetic acid aqueous solution or 2.0 w/v % alginate in 1.0 v/v % acetic acid aqueous solution. The chitosan hydrogels swelled in 1.0 v/v % acetic acid aqueous solution and shrank when incubated in the presence of alginate, validating that this approach is applicable to hydrogels formed by polycationic polymers as well. Figure if visually shows the swollen chitosan hydrogel (2.0 w/v %) in 1.0 v/v % acetic acid aqueous solution (lower construct) and the shrunken hydrogel (upper construct) in 2.0 w/v % alginate in 1.0 v/v % acetic acid aqueous solution when the molar ratio of glutaraldehyde to chitosan chains was 5:1. With increasing amounts of glutaraldehyde, lower swelling in acetic acid solution and lower shrinking in alginate solution were observed, which can be ascribed to the removal of the positive charges in chitosan by the reaction of the primary amines with glutaraldehyde.

### Shrinking Behaviors of Extrusion-Printed Structures

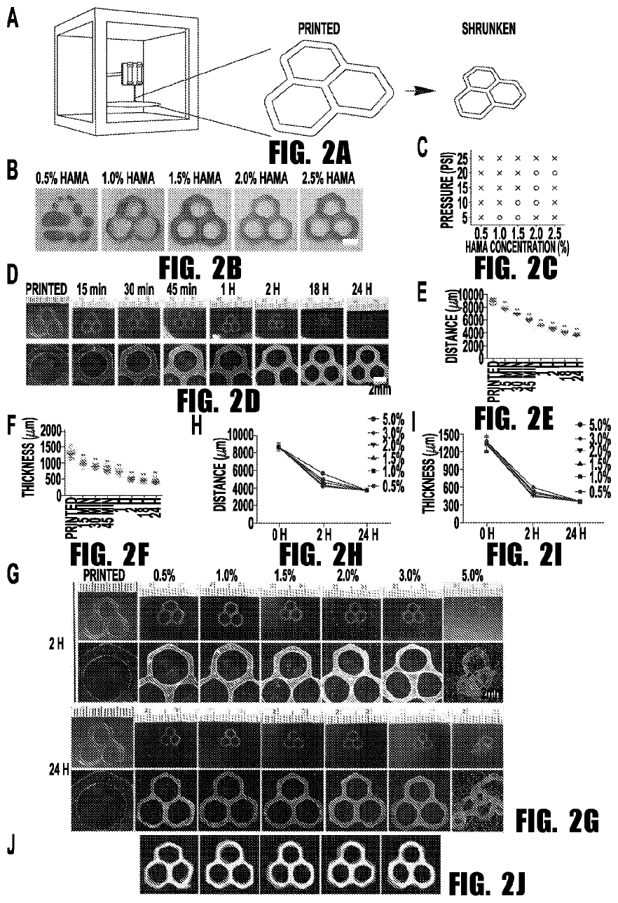

Here, we sought to demonstrate that the same process might be adapted to the printed hydrogel structures to achieve resolution improvement otherwise only possible through changes in printing parameters (such as nozzle size, pressure, and/or nozzle moving speed). We first evaluated the printability of HAMA inks (0.5-2.5 w/v %) in extrusion printing by depositing hexagonal patterns (FIG. 2a). Our printability mapping (FIG. 2b, c) suggested an optimal HAMA ink formulation of 2.0 w/v %, which was chosen for the subsequent experiments using extrusion printing. Young's moduli of the resulting HAMA hydrogels after photocrosslinking were evaluated, where the values ranged from 1.7±0.2 to 24.1±3.3 kPa. These values are in accordance with those reported in literature for HAMA hydrogels. Rodell et al., Adv. Mater. 28, 8419-8424 (2016).

We immersed our printed structures in 2.0 w/v % HMw chitosan dissolved in 1.0 v/v % acetic acid in deionized water (pH=4.7). It was observed that the hexagonal HAMA hydrogels shrank to smaller sizes as a function of time. As illustrated in FIG. 2d-f, the distance (side-to-side) of the hexagons was reduced from 893±377 to 3674±163 μm in 24 h, or 41.1% of the original size (FIG. 2e). The thickness of the fibers in the hexagons also decreased from 1295±113 to 424±68 μm, or 32.7% of the original size (FIG. 2f). The Young's modulus of HAMA increased from 15.9±1.8 kPa to 24.3±3.0 kPa in the 2.0 w/v % HAMA group. Shrinking of the HAMA hydrogels was also validated by measuring their volume changes, where the 2.0 w/v % HAMA hydrogel shrank to ˜20% of the original volume after chitosan complexation for 24 h. We subsequently investigated how different concentrations of HMw chitosan (0.5, 1.0, 1.5, 2.0, 3.0, or 5.0 w/v %) dissolved in 1.0 v/v % acetic acid affected the shrinking of the printed HAMA (2.0 w/v %) structures. As revealed in FIG. 2g-i, at 2 h of shrinkage, the side-to-side distances of the hexagons decreased to a range between 4144±98 μm (3.0 w/v % of HMw chitosan) and 5664±91 μm (0.5 w/v % of HMw chitosan), or 47.6-65.1% of the original size. After 24 h of immersion, the side-to-side distance of the hexagons was further reduced to −3700 μm, while the thickness of the fibers shrank from the initial 1350 μm to 360 μm. Distinct from those at 2 h of shrinking, neither the distance of the hexagons nor the thickness of the fibers indicated significant differences among the concentrations of HMw chitosan, suggesting that the higher concentration of chitosan led to faster shrinking but the final gel volume could be reached in 24 h or earlier. It should be noted though, that the printed structures in the 5.0 w/v % HMw chitosan group became irregular likely due to the high viscosity of the chitosan solution at this concentration that prevented uniform shrinking of the patterns. Since the fidelity of shrinking is important for retaining the printed structures, here we visualized the distortions of the 2-h shrinking and 24-h shrinking images by superimposing the 2-h shrinking images on top of the corresponding 24-h shrinking images. As shown in FIG. 2j, the distortions (indicated by background grids) were deemed to be within an acceptable range, indicating that only little deformation of the total structure had taken place during the shrinking.

Considering that chitosan was dissolved in 1.0 v/v % acetic acid at a pH value of 4.7, we subsequently evaluated the possibility of utilizing Q. chitosan, which easily dissolves in deionized water and aqueous solutions at physiological pH, as also used for shrinking non-printed hydrogel constructs above. As a control, we initially dissolved Q. chitosan in 1.0 v/v % acetic acid aqueous solution and investigated the shrinking behavior of the printed 2.0 w/v % HAMA structures. The results were similar to those obtained with regular HMw chitosan solutions. The shrinking rate was proportional to the concentration of Q. chitosan and reached equilibrium at or before 24 h. Remarkably, the hexagons in 5.0 w/v % Q. chitosan were able to retain their shape and shrank to 40.8±1.8% in side-to-side distance and 24.1±2.6% in thickness as compared to the original dimensions, since the viscosity of Q. chitosan solution was much lower than that of the chitosan solution at this concentration, which facilitated uniform shrinking. When we shifted to the use of Q. chitosan in de-ionized water, the rate of shrinking seemed to become slightly slower as compared to using Q. chitosan or HMw chitosan in acetic acid aqueous solution, where the side-to-side distance was reduced to 62.0-99.3% and the thickness to 52.1-99.5% of the original constructs after 2 h of incubation. Nevertheless, when the time was extended to 24 h, hydrogel sizes shrank down to 41.2-56.4% for the side-to-side distances and 33.0-45.9% for the thicknesses, both similar to the degrees of shrinking with HMw chitosan or Q. chitosan in 1.0 v/v % acetic acid aqueous solution. Moreover, the distortions of the 2-h shrinking and 24-h shrinking images were also visualized.

To illustrate that our method truly works in all three spatial dimensions, we further printed a HAMA (2.0 w/v %) pyramid frame in a gelatin supporting matrix (1.5 w/v %) followed by UV crosslinking. After photocrosslinking of HAMA, the gelatin was heated and stepwise replaced with water. Finally, the 3D-printed pyramidal frame structure was incubated in 1.0 w/v % HMw chitosan in 1.0 v/v % acetic acid aqueous solution for 24 h to achieve complete shrinking. The printed HAMA pyramid shrank uniformly in every direction (length, height, thickness), to about 60% in linear dimensions those of the original constructs.

To eliminate the effect caused by changes in pH, we further incubated the samples in aqueous solutions of HMw chitosan and Q. chitosan (both dissolved at 2.0 w/v % in 1.0 v/v % acetic acid aqueous solution), or an aqueous solution of pH=1.0 (adjusted using perchloric acid without chitosan). The volume of the HAMA hydrogel in perchloric acid was still significantly larger than those incubated in the chitosan solutions, verifying that the mechanism of shrinking with chitosan was not purely related to neutralizing the negative charges on the HAMA molecules. The Young's moduli post-shrinking as expected, showed an opposite trend with their volumes. We next evaluated the shrinking behavior of the printed HAMA structures (2.0 w/v %) in acetic acid aqueous solutions of different pH values. During the experiments, we found that the sizes of the printed HAMA hexagons shrank in acetic acid solutions of different pH values, where the lower the pH, the smaller the size, which yet were still larger than those shrunken with 2.0 w/v % HMw chitosan in 1.0 v/v % acetic acid aqueous solution. The pH-induced shrinking was reversible, initially causing structures incubated in acid to shrink but recovering to the original printed size when subsequently placed into de-ionized water. In contrast, the chitosan-driven shrinking was stable during the investigated time frame attributed to the complexation process, consistent with our stability analysis on the bulk HAMA constructs. Therefore, the neutralization of charges by changes in pH could lead to a certain degree of shrinkage, but charge compensation by polyelectrolyte complexation of the polyanionic polymers and the polycationic hydrogels resulted in irreversible shrinkage in an electrostatic binding strength-dependent manner (see FIG. 1d).

Interestingly, we found that the printed HAMA structures could shrink in cell culture medium as well. When subjecting the HAMA hydrogels to Q. chitosan dissolved in Dulbecco's modified Eagle medium (DMEM, 2.0 w/v %), the constructs shrank to approximately half of their thickness and about 75% in size-to-size distance, although the shrinking rate was slower compared to those in acid or in water. This observation could be attributed to the higher ionic strength of DMEM (˜170 mM) as compared to the 0-20 mM found in deionized water, where ionic strength of the medium dampens electrostatic interactions between positive and negative moieties. Nevertheless, our results indicated that shrinking of the printed constructs in the presence of cells is possible.

Printed alginate hexagons (2.0 w/v %) were physically crosslinked by 0.3-M CaCl₂), after which they were shrunken in 1.0 v/v % acetic acid aqueous solution, or in 2.0 w/v % chitosan of different molecular weights and types in 1.0 v/v % acetic acid aqueous solution. Finally, we printed chitosan hexagons (2.5 w/v %), which were subsequently crosslinked by glutaraldehyde (800 μM). As expected, the printed chitosan hexagons shrank in 2.0 w/v % alginate in 1.0 v/v % acetic acid aqueous solution.

### Shrinking Behaviors of Sacrificially Printed Microchannel-Embedded Constructs

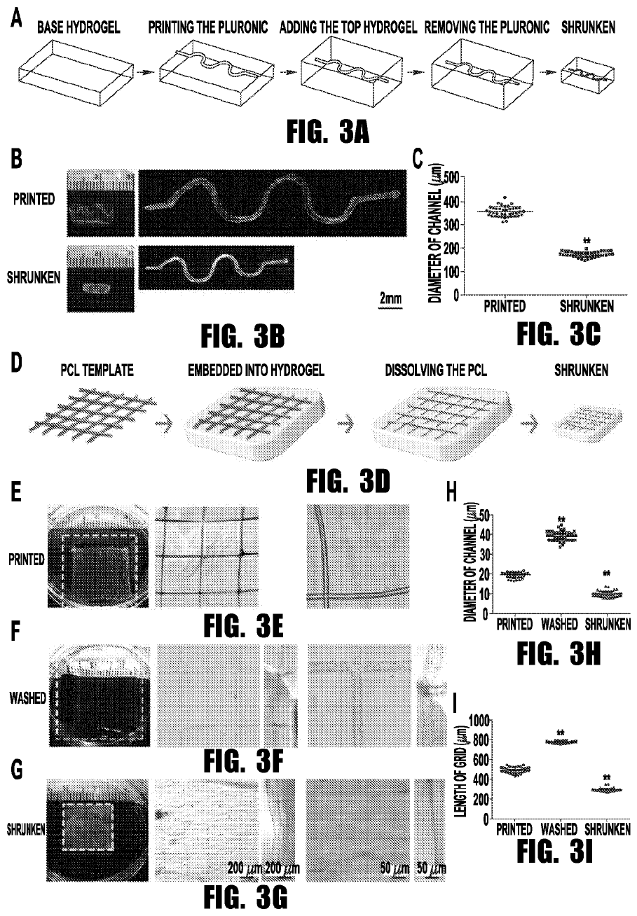

Once we validated the hypothesis that electrostatic complexation could enable efficient size reduction of extrusion-printed HAMA patterns, we explored if the same concept can be applied to other commonly used 3D printing strategies. Again, we aimed to achieve improved resolution without needing to adjust the printing hardware or parameters. Among all approaches, sacrificial printing is a frequently used method to generate hollow, perfusable microchannels within hydrogel constructs. These microchannels would serve as biomimetic cannular structures to emulate human tissues, such as the vasculature or the proximal tubules found in kidneys. A Pluronic F127 solution is thermosensitive, meaning that it flows at low temperatures but forms a hydrogel at elevated temperatures. As such, it has been frequently used as a fugitive ink, serving as a template during printing that can later be selectively removed from the hydrogel matrices to create hollow microchannels (FIG. 3a). We produced HAMA hydrogel constructs with a channel (2.0 w/v %) using Pluronic-based sacrificial printing (FIG. 3b). Measuring the diameter of the microchannels after removing the Pluronic fugitive template showed a value of 355±21 μm, in accordance with those previously fabricated under similar conditions. Lin et al., Proc. Natl Acad. Sci. USA 116, 5399-5404 (2019). When the entire constructs were immersed in 2.0 w/v % HMw chitosan in 1.0 v/v % acetic acid aqueous solution for 24 h, a gel size reduction was observed with associated decrease in the microchannel diameter to 174±12 μm, or 49.0±3.4% of the original size (FIG. 3b, c). The distortions between pre-shrinking and post-shrinking patterns were visualized and showed negligible changes in original shape.

While sacrificial printing using Pluronic as the fugitive ink renders the fabrication of hydrogel-embedded microchannels convenient, the channel sizes are typically limited to a few hundred micrometers or larger. Although with our unique shrinking strategy we could further reduce the channel diameter by a factor of 2, creating microchannels in the sub-100 μm scale with conventional sacrificial printing, relying on fugitive hydrogel inks is still challenging. More recently, melt electrospinning writing (MEW) has attracted increasing attention due to its ability to deposit well-defined meshes with filament sizes ranging from few micrometers down to sub-micron levels. Hochleitner et al., Biofabrication 7, 035002 (2015). To further demonstrate the utility of our shrinking method, we manufactured MEW-printed polycaprolactone (PCL) grids and embedded them in HAMA hydrogels (2.0 w/v %). After UV-crosslinking, we selectively dissolved the PCL templates, and subjected the microchannel-containing HAMA constructs to shrinking (FIG. 3d). As revealed in FIG. 3e, the pristine MEW meshes embedded in the hydrogels were 20±1 μm in diameter with an interfiber distance of 490±29 μm. The microchannels created by sacrificing the MEW templates following washing and swelling were 39±2 μm in diameter, and the distance between the adjacent microchannels (length of grid) was 772±10 μm (FIG. 3f). After immersing in 2.0 w/v % HMw chitosan in 1.0 v/v % acetic acid aqueous solution for 24 h, the microchannels shrank down to 10±2 μm (˜50.9% of original size, FIG. 3g, h), which was comparable to the diameter of single capillaries. In addition, the grid length was reduced to 292±16 μm (˜59.5% of the original size, FIG. 3g, i) with an associated decrease in the overall volume of the HAMA constructs. As such, using our shrinking method, the sacrificial MEW fibers of tens of micrometers could also be used to still generate sub-micron microchannels without needing the ultrafine fibers to start with.

Shrinking behaviors of coaxially printed cannular constructs. Human tissues contain various cannular structures, such as the blood vessels for transporting cells, nutrients, and waste, the lymphatic vessels for draining fluids, and the tubules present in the kidney for secretion and reabsorption functions. While sacrificial printing allows for emulation of matrix-embedded microchannels, microfluidic printing enabled by the adoption of coaxial, concentric printheads has featured single-step fabrication of standalone cannular structures. Both others (Datta et al., Acta Biomaterialia 51, 1-20 (2017)) and our lab (Jia et al., Biomaterials 106, 58-68 (2016); Pi et al., Adv. Mater. 30, e1706913 (2018)) have previously reported similar techniques in producing perfusable cannular tissues, where the minimum diameters of the obtained microfibers were limited to no smaller than a couple hundred micrometers due to the physical constraints of the sizes of the multilayered nozzles.

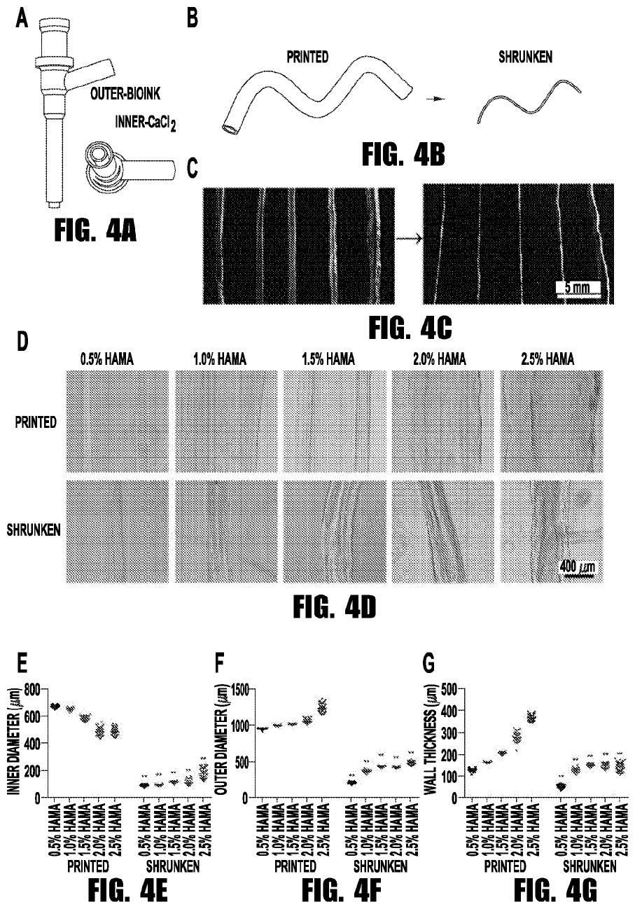

A coaxial printhead was thus designed and optimized to print HAMA-based tubes (FIG. 4a, b). Different from direct extrusion printing and sacrificial printing, we mixed HAMA (0.5-2.5 w/v %) with alginate (0-2.0 w/v %) of different concentrations for use as the inks, to accommodate the microfluidic printing needs. The inks were delivered from the outer layer of the coaxial printhead, whereas the crosslinking CaCl₂ solution (0.3 M) was carried from the interior. The printability of the inks was first examined. It was observed that, when the concentrations of HAMA ranged from 0.5 to 1.0 w/v % and the alginate was in the range of 0.5-2.0%, the tubes were smoothly printed and uniform in shape (FIG. 4c, d). The cannular structures became non-uniform when the concentration of HAMA was increased to higher than 1.0 w/v %. The optimal ink composition consisted of 1.0 w/v % HAMA, 0.5 w/v % alginate and 0.5 w/v % photoinitiator, and was used throughout the subsequent experiments.

The inner diameters of the as-printed tubular structures were measured at 670±10 μm (0.5 w/v % HAMA), 648±10 μm (1.0 w/v % HAMA), 583±16 μm (1.5 w/v % HAMA), 478±28 μm (2.0 w/v % HAMA), and 483±29 μm (2.5 w/v % HAMA) (FIG. 4e-g). The tubes were then subjected to the same shrinking process (2.0 w/v % HMw chitosan in 1.0 v/v % acetic acid aqueous solution for 24 h) we here established. After shrinking, the inner diameters narrowed down by 3-8 times of their original sizes to 90±7 μm, 93±4 μm, 115±7 μm, 108±19 μm, and 176±41 μm, respectively (FIG. 4e). Their outer diameters were decreased to 20.7-42.0% of original ones as well (FIG. 4f), so did the wall thicknesses (38.7-79.1%, FIG. 4g). We also compared the ratios obtained by the outer diameter divided by the inner diameter (OD/ID) before and after shrinkage, as well as the ratios of the outer diameter divided by the wall thickness (OD/WT), as measurements of fidelity of shrinkage in the printed cannular structures. Before shrinkage, the ratios of OD/ID were 1.38±0.02 (0.5 w/v % HAMA), 1.50±0.02 (1.0 w/v % HAMA), 1.71±0.04 (1.5 w/v % HAMA), 2.16±0.13 (2.0 w/v % HAMA), and 2.52±0.03 (2.5 w/v % HAMA) for the respective tubes. After shrinkage, the ratios increased to 2.15±0.22, 3.78±0.26, 3.64±0.23, 3.85±0.60, and 2.73±0.58, respectively. As for the OD/WT, there were decreases comparing to the as-printed cannular structures, from 7.25±0.33, 5.97±0.13, 4.83±0.15, 3.74±0.23, and 3.32±0.03 to 3.81±0.35, 2.73±0.07, 2.76±0.06, 2.73±0.19, and 3.30±0.47, respectively. These metrics can guide the designs of cannular structures before shrinking according to the needs of the final desired sizes post-shrinking.

A printhead constructed with smaller needles (inner: 30 G, outer: 18 G) was used to print thinner HAMA/alginate tubes, which possessed an inner diameter, outer diameter, and wall thickness of 301±3, 438±6, and 68±3 μm, respectively. Following shrinking, the sizes were reduced to 37±3, 148±14, and 56±7 μm, respectively. This set of values is close to the sizes of small blood vessels (arterioles and venules, 8-100 μm), lymphatic capillaries (30-80 μm), and proximal tubules (50-60 μm), making them physiologically relevant. The changes in the volumes and Young's moduli of constructs formed with inks containing different concentrations of HAMA but constant 0.5 w/v % alginate before and after shrinking in 2.0 w/v % HMw chitosan were measured, as well as for HMw chitosan and Q. chitosan (both dissolved at 2.0 w/v % in 1.0 v/v % acetic acid aqueous solution) or in a perchloric acid solution of pH=1, and similar trends were found as reported for the HAMA hydrogels earlier.

### Shrinking Bioprinting for Applications Involving Cells

We endeavored on two aspects that are relevant to the future applications of this shrinking printing strategy in cell cultures, i.e., to expand its conceptual feasibility to a more bioactive ink of GelMA, and to prove the concept that the density of embedded cells in a sacrificially bioprinted construct may be increased through the shrinking process without significantly affecting their viability.

We first investigated whether our shrinking concept could be extended to GelMA, which is a gelatin derivative featuring intrinsic cell-binding moieties and is capable of on-demand photocrosslinking. Loessner et al., Nat. Protoc. 11, 727-746 (2016). GelMA solutions or hydrogels also exhibit a net negative charge under neutral or slightly acidic pH values (Shirahama et al., Sci. Rep. 6, 31-36 (2016)), and we therefore hypothesized that an environment rich of cationic polymers would also shrink bioprinted GelMA constructs. Indeed, GelMA constructs sacrificially bioprinted to contain microchannels of 612±32 μm in diameter through the fugitive Pluronic F127 ink shrank to 55.6±9.1% of the original size (341±56 μm) when immersed in 2.0 w/v % chitosan in 1.0 v/v % acetic acid aqueous solution, and the distortions were deemed to be in an acceptable range.

To demonstrate the versatility of our shrinking technology, we further used a modified embedded sacrificial printing method to generate microchannels within GelMA/HAMA hydrogel constructs using gelatin as the fugitive ink, and subsequently illustrated their ability to be shrunken (using 2.0 w/v % HMw chitosan dissolved in 1.0 v/v % acetic acid aqueous solution) and perfused. Consistent with other reports, it was shown that endothelial cells could be populated on the surfaces of the microchannels when seeded post-shrinking, indicating the reasonably good biocompatibility of the GelMA/HAMA-chitosan matrix after complexation. In addition, GelMA constructs made from MEW-PCL templates through washing out the PCL shrank from 19±1 to 12±2 μm, or ˜62.1% of original size, whereas the length of grid was reduced from 503±26 μm to 333±16 μm, or ˜66.3% of the original dimension.

In microfluidic bioprinting, the inner diameter of the resultant GelMA (5.0 w/v %)/alginate (0.5 w/v %) cannular constructs decreased from 313±8 μm prior to shrinking to 39±4 μm afterwards, which was 12.5% of its original size or a factor 8 in reduction. The outer diameter and the wall thickness also became proportionally smaller. The shrinking results were comparable to those with bioprinted HAMA constructs.

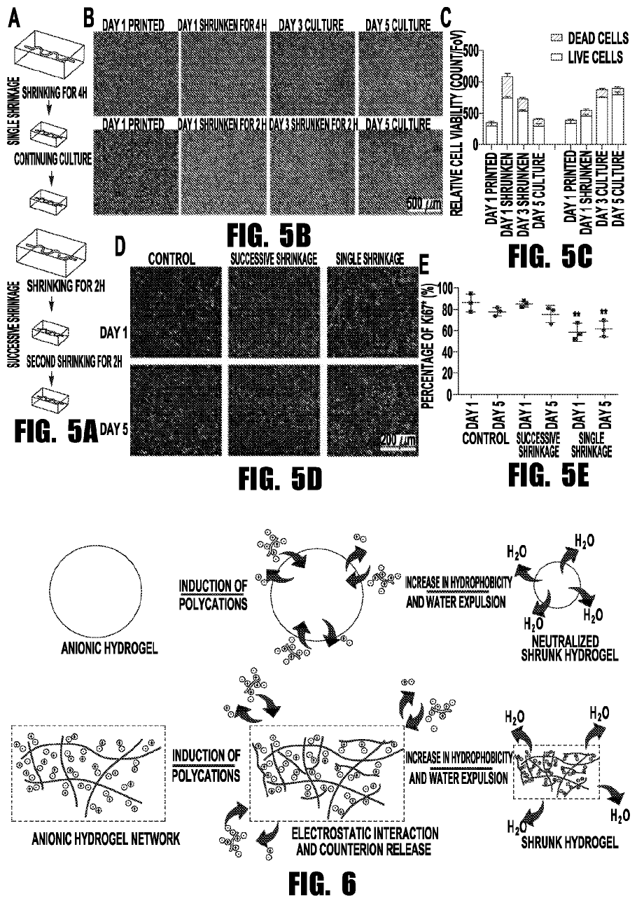

We subsequently explored the cytocompatibility of the shrinking method, where the Q. chitosan solution at physiological pH was used as the shrinking agent. A mixture of GelMA (2.5 w/v %) and HAMA (0.5 w/v %) was adopted as the ink to improve the bioactivity of the hydrogel constructs over those made from pure HAMA. We designed two shrinking processes and compared their effects on size reduction and cell viability. The first procedure consisted of a single shrinkage step (FIG. 5a), i.e., a hydrogel construct encapsulating MCF-7 breast cancer cells, was left in 1.0 w/v % Q. chitosan in culture medium for 4 h, while in the second procedure termed successive shrinkage, we shrank the same type of construct twice in the same shrinking agent each time for 2 h, on the 1st day and the 3rd day (FIG. 5b).

Through live/dead staining, we found that the cell density was significantly elevated after 4 h of shrinkage (single shrinkage; 1080±49 total cells per field of view [FoV], i.e., 1417 by 1417 μm²), compared to shrinkage for only 2 h (549±40 total cells per FoV), on the 1^{st} day (FIG. 5b, c). In contrast, the original cell density was calculated to be in between 350 and 400 total cells per FoV. Nevertheless, a large number of cells were dead in the single shrinkage process likely due to the prolonged exposure to the shrinking agent. On the 3rd day, the samples in the successive shrinkage group were shrunken again for another 2 h, which brought the cell density up to 883±32 total cells per FoV. It should be noted that, although this density was lower than that of the single-shrinkage method on the 1st day, when we counted only viable cells, the values were in fact similar (748±19 versus 747±29 live cells per FoV), indicating that two successive size reductions each at a lower degree could minimize the harm of the process to the embedded cells and maintain their proliferation potential. Indeed, this trend became more pronounced during longer cultures. The viability of MCF-7 cells on the 5th day in the successive shrinking group (905±49 total cells per FoV, percentage of live cells: 88.1±4.7%, or 797±42 live cells) was much higher than those in the single shrinkage group (406±32 total cells per FoV, percentage of live cells: 73.8±8.9%, or 297±36 live cells). Ki67 was also assessed as a proliferation marker to show proliferation before and after shrinkage in GelMA/HAMA constructs. As revealed in FIG. 5d, e, the percentage of Ki67+ stained nuclei in the control group at the 1st day was 86.1±6.5%, and for the cells in the successive shrinkage group, the positive rate was 84.9±2.6%, similar to the control group. In contrast, it significantly decreased to 58.5±8.0% when single shrinkage for 4 h was adopted. After 5 days of culture, the percentage of Ki67+ stained nuclei were 77.5±4.0, 75.3±8.0, and 61.4±7.6% in the control group, successive shrinkage group, and single shrinkage group, respectively. Interestingly, the MCF-7 cells that underwent two successive shrinkages still exhibited a good proliferative potential comparable to the control group and higher than the single shrinkage group at the same time.

The method was extended to several other cell types including the C2C12 mouse skeletal muscle cells, which maintained satisfactory viability post-shrinking. The C2C12 cells spread well in the as-printed GelMA/HAMA hydrogel constructs, and after 2 h of shrinkage the density of the cells was doubled although their sizes seemed to have decreased possibly caused by the shrinking process. However, following 3 days of culture, the cells were able to spread again and proliferated throughout the subsequent culture period.

We noted that however, while MCF-7 cells and C2C12 cells performed reasonably well after shrinkage, another cell type that we examined, i.e., human umbilical vein endothelial cells (HUVECs), appeared to be much more sensitive to the shrinking processes. The percentage of Ki67+ stained nuclei was analyzed as a proliferation marker; at the 5th day of culture, only 42.2±3.0% (successive shrinkage group) and 31.1±2.1% (single shrinkage group) of HUVECs were Ki67+ stained, significantly lower than the control group at the 1st day of culture. We reason that such observations might be relevant to the differential sensitivities of the different cell types to the shrinking agent, Q. chitosan, for which we measured the metabolic activity of MCF-7 cells and HUVECs exposed to Q. chitosan PBS solutions at different concentrations for 30 min, 2, 4, and 24 h. The concentrations reflecting 50% reduction in cell metabolic activity, depicted as toxic concentration (TC)₅₀ values of these two cell types were calculated. Indeed, the TC₅₀ values for MCF-7 cells at 30 min, 2, 4, and 24 h of Q. chitosan treatment were 0.505, 1.487, 0.371, and 0.131 mg mL-1, respectively, whereas those for HUVECs were significantly lower at all time points at 0.002, 0.077, 0.002, and 0.009 mg mL⁻¹, respectively. These results suggested that HUVECs are remarkably more sensitive to Q. chitosan than MCF-7 cells, explaining their pronounced reduction in proliferation potential during the shrinking conditions that we used, even in the case of two successive shrinkage. We anticipate that these observations will provide insights towards selection of shrinking agent concentrations for sensitive and resistant cells in the future.

We finally demonstrated the feasibility of shrinking sacrificially printed hydrogels in the presence of cells. GelMA/HAMA constructs containing green fluorescent protein-labeled HUVECs (GFP-HUVECs) were produced with the Pluronic fugitive inks, and subjected to the two different shrinking procedures. In the single shrinkage group, the microchannels inside the block were reduced to 64.2±3.5% of their initial diameters. In the successive-shrinkage group, the microchannels shrank to 74.9±5.8% after the first shrinkage, and after another shrinkage on the 3rd day, the diameter of the microchannels was reduced to 58.3±7.3% of its original size, similar to the case of single shrinkage and within the physiological range of small blood vessels. As expected, the loss of the GFP signals was more pronounced in the single shrinkage group.

### Discussion

In conclusion, we report here a printing strategy of complexation-induced resolution enhancement, i.e., shrinking printing, through post-treatment of the printed structures without changing the printer hardware or printing parameters. We conducted our proof-of-concept studies with several techniques of printing and succeeded in all cases, including direct extrusion printing, sacrificial printing, and microfluidic hollow tube printing. Notably, our data showed that these printed constructs could reduce in their sizes by different degrees, comparing to their original dimensions. In addition, results indicated that this method is broadly applicable, i.e., a printed anionic hydrogel structure might be shrunken by a cationic polymer, or vice versa. We finally demonstrated that successive shrinking could preserve, in a cell type-dependent manner, the viability of cells embedded in the printed hydrogel matrices compared to a single, longer shrinking procedure, revealing the potential applications of our shrinking printing method towards tissue biofabrication. We therefore anticipate widespread adoption of our unique technology in future printing of hydrogel constructs for various application areas with further optimizations.

### Methods

Synthesis and characterizations of HAMA. Hyaluronic acid was functionalized with methacrylate groups through a transesterification reaction with methacrylic anhydride. Burdick., J & Prestwich, G., Adv. Mater. 23, H41-H56 (2011). In a typical synthesis, 3 g of hyaluronic acid sodium salt (Mw: 1530 kDa, Lifecore Biomedical, USA) was dissolved overnight in 400 mL of de-ionized water at 4° C. The solution was placed on ice and an equal volume of dimethyl formamide (DMF, Sigma-Aldrich, USA) was added whilst stirring vigorously. Methacrylic anhydride (MA, Sigma-Aldrich) was added in a 5:1 molar ratio of MA (5.8 g, 37.5 mmol) to hyaluronic acid disaccharide units over the course of 4 h using a syringe pump (1.375 mL h⁻¹). During these 4 h, the pH was controlled using a Mettler DL21 titrator (Mettler-Toledo, The Netherlands) connected to a pH meter, which dispensed an aqueous 0.5M NaOH (Sigma-Aldrich) solution whenever the pH of the solution dropped below 8.5. After complete addition of the methacrylic anhydride, pH was monitored for an additional hour and maintained above pH 8.5. Subsequently, the reaction mixture was left at 4° C. overnight. The next day, NaCl (Sigma-Aldrich) was dissolved in the reaction mixture to up to 0.5M and the mixture was precipitated in 10 equal volumes of ethanol at −78° C. (cooled with an acetone dry ice bath). HAMA was collected as dry white pellets, dissolved in deionized water and dialyzed against de-ionized water for 2 days to remove impurities (Visking, regenerated cellulose dialysis membrane, molecular weight cut off [MWCO]: 12-14 kDa, VWR, The Netherlands). After dialysis the HAMA solution was freeze-dried to yield a white powder.

Synthesis and characterizations of FITC-Q. chitosan. Quaternized chitosan was fluorescently labeled using FITC (Sigma-Aldrich). In short, 1 g of Q. chitosan was dissolved overnight in 200 mL of freshly made sodium carbonate buffer (pH=9.0). Subsequently, a fresh solution of 10 mg of FITC in 10 mL of dry dimethylsulfoxide (DMSO, Sigma-Aldrich) was added dropwise to the Q. chitosan solution under vigorous stirring. The reaction was left to proceed for 4 h. Both the dissolution FITC in DMSO solution and the reaction with Q. Chitosan were performed in a dark environment to limit potential photodegradation. Upon completion of the reaction, the solution was directly poured into dialysis bags (MWCO: 10-14 kDa) and dialyzed in de-ionized water for 6 days whilst protected from light. Finally, the dialyzed solution was freeze-dried overnight to yield FITC-Q. chitosan as an orange powder.

The degree of substitution of FITC onto the Q. chitosan was studied using UVvis spectrometry, where absorbances were measured at 280 (A280) and 488 (A488) nm, respectively. Molar extinction coefficients of 175 and 68 Mcm⁻¹ were used for Q. Chitosan (Fc) and FITC (FFITC), respectively. It was found that one mole of Q. chitosan was conjugated with 29.7 mol of FITC.

Synthesis and characterizations of GelMA. GelMA was synthesized by reaction of type B bovine skin gelatin (˜225 bloom, Sigma-Aldrich) with methacrylate anhydride at 50° C. for 1 h in PBS (pH=7.4, Gibco, USA). Van Den Bulcke et al., Biomacromolecules 1, 31-38 (2000). Methacrylic anhydride was added dropwise in a 0.6:1 weight ratio of anhydride to gelatin. Next, the solution was diluted 1:1 with de-ionized water and dialyzed for 5 days (dialysis membrane as used for the HAMA synthesis), and subsequently freeze-dried to yield a white powder.

Determination of degree of methacrylation and methacrylate conversion. The degree of methacrylation (DM) for the synthesized HAMA was determined using a previously developed high-performance liquid chromatography (HPLC) method. Stenekes, R. J. H. & Hennink, W. E., Polymer 15, 5563-5569 (2000). In short, 15 mg of polymer or dried hydrogel was dissolved overnight at room temperature in 6 mL of aqueous 0.02-M NaOH solution. Next, 1 mL of acetic acid was added and the samples were injected into an Alliance Waters HPLC system equipped with UV-VIS detection monitoring at 210 nm (Dual Lambda Absorbance, USA) and a Sunfire C18 column (column temperature: 50° C.). An isocratic method was used based on eluent consisting of 15:85 acetonitrile: de-ionized water (pH=2, adjusted with perchloric acid) with a set flow of 1 mL min⁻¹. The samples were referenced to a calibration curve of known concentrations of methacrylic acid. Concentrations were then calculated to yield the DM, defined as the number of methacrylate groups per 100 disaccharide units. The DM of HAMA was found to be 28.8±0.4% (n=3).

The DM of GelMA was defined as the number of methacrylate groups per available lysine found in the gelatin and was determined by ¹H NMR in D₂O. Eva Hoch et al., J. Mater. Chem. B 1, 5675-5685 (2013). The DM of GelMA was found to be approximately 50%.

Preparation and printability of the HAMA inks. For the inks used in extrusion printing and sacrificial printing, various concentrations (w/v) of HAMA (0.5%, 1.0%, 1.5%, 2.0%, or 2.5%) were dissolved in de-ionized water in room temperature overnight. In addition, 0.5 w/v % photoinitiator (Irgacure 2959; Sigma-Aldrich) was added to initiate photocrosslinking upon UV-irradiation (approximately 10 mW cm⁻², 360-480 nm, 40 s). For the blend inks for coaxial printing, different concentrations of HAMA (0-3.0 w/v %) and alginate (0-2.0 w/v %; lot number: BCBP9590V, Sigma-Aldrich) were evaluated, where the final formulations were determined to be 0.5, 1.0, 1.5, 2.0, or 2.5 w/v % HAMA+0.5 w/v % alginate+0.5 w/v % photoinitiator. Inks were prepared and stored at 4° C. until use.

Preparation of GelMA inks. For the inks used in sacrificial printing, the GelMA was dissolved in de-ionized water in 37° C. for 1 h at a concentration of 5.0 w/v %. In addition, 0.5 w/v % photoinitiator was added to enable crosslinking. In the blend ink used for coaxial printing, the final formulation was 5.0 w/v % GelMA+0.5 w/v % alginate+0.5 w/v % photoinitiator. Inks were stored at 4° C. until use.

Preparation of chitosan solutions and shrinking efficiency in different chitosan solutions. Four types of chitosan were used, where three of different molecular weights (LMw: 15 kDa; MMw: 50-190 kDa; HMw: 700-800 kDa) were of similar degree of deacetylation (85%) and from Golden-Shell, China. Q. chitosan (Mw: 50-100 kDa), which was ˜90% deacetylated and subsequently 95% quaternized to yield permanent positive charges, was from Cool Chemistry, China. They were dissolved at a concentration of 2.0 w/v %, in 1.0 v/v % acetic acid aqueous solution when comparing the effect of chitosan of different molecular weights and types. Another three types of chitosan with different degrees of deacetylation (72.5%, 77.8%, and 94.6%; Heppe Biomedical, Germany) were used to study the effects of shrinking agent deacetylation. All chitosan types were of similar molecular weights (50-250 kDa), and were all dissolved at a concentration of 2.0 w/v % in 1.0 v/v % acetic acid aqueous solution for usage. 1.0 w/v % HAMA hydrogels were fabricated and immersed in these shrinking agents for 24 h and the volumes before and after shrinking were measured.

Various concentrations (0.5, 1.0, 1.5, 2.0, 3.0, or 5.0 w/v %) of HMw chitosan and Q. chitosan were dissolved in 1.0 v/v % acetic acid aqueous solution, de-ionized water, or DMEM (Gibco) at 37° C. The solutions were vortexed and stored at 4° C. Before use, they were pre-heated to 37° C. The printed hexagons were immersed in HMw chitosan (0.5, 1.0, 1.5, 2.0, 3.0, or 5.0 w/v %) dissolved in 1.0 v/v % acetic acid aqueous solution, or Q. chitosan (0.5, 1.0, 1.5, 2.0, 3.0, or 5.0 w/v %), which was dissolved in 1.0 v/v % acetic acid aqueous solution, de-ionized water, or DMEM. The shrinking processes were recorded at 2 and 24 h microscopically and through photography, and measurements were made using imageJ (National Institutes of Health, USA).

Preparation of hydrogel discs and dimension measurements. The anionic polymer solutions (2.0 w/v % HAMA, 2.0 w/v % alginate) were cast in cylindrical PDMS molds (D=4.5 cm) and subsequently gelled through either exposure to UV irradiation (HAMA) or a CaCl₂ solution (alginate), or both. Hydrogels that were gelled with CaCl₂ were washed with de-ionized water to remove excess ions. Subsequently, biopsy punches (Integra Miltex, The Netherlands) were used to produce cylindrical hydrogels (D=6 mm, H=2 mm, ˜56.5 μL or D=8 mm, H=3.4 mm, ˜170.9 μL).

The cationic polymer (HMw chitosan) solution was prepared in 1.0 v/v % acetic acid in water at concentrations of 2.0 w/v %. The HMw chitosan solution was then cast into the PDMS molds, and subsequently, 2 mL of glutaraldehyde (with concentrations of 200, 400, 800, or 2400 μM) in 1.0 v/v % acetic acid solution was gently pipetted on top. The crosslinking reaction was left to proceed for 1 h, biopsy punch was used to produce cylindrical hydrogels (D=6 mm, H=2 mm, ˜56.5 μL). The hydrogels were incubated in a perchloric acid solution (pH=1.0), 1.0 v/v % acetic acid aqueous solution (pH=4.7), or 1.0 v/v % acetic acid aqueous solution with 2.0 w/v % of the shrinking agent. For all hydrogel shrinking studies, measurements before and after shrinking were performed using Vernier calipers (measurement error˜30 μm).

Measurements of mechanical properties. To measure the Young's modulus and volume of HAMA hydrogels with/without alginate before and after shrinkage, crosslinked hydrogel disks (D=6 mm, H=2 mm, ˜56.5 μL) were prepared as mentioned before, incubated for 24 h in HMw chitosan, Q. chitosan (both dissolved up to 2.0 w/v % in 1.0 v/v % acetic acid aqueous solution), or an aqueous solution of perchloric acid adjusted to pH 1.0. Moreover, the hydrogels containing alginate were briefly incubated in a CaCl₂ solution before shrinking. Compression tests were performed in triplicate on a 2980 DMA (TA Instruments, the Netherlands) with a ramp of 2.0 N min⁻¹ up to a maximum of 18.0 N. The elastic modulus was calculated as the slope of the start of the stress-strain curve that was obtained from the compression test. Specifically, we used the linear region between 10 and 30% strain.

Stability studies of the shrunken hydrogels. The following hydrogel formulations were used to study the stability of the shrinking effect, i.e., 2.0 w/v % HAMA, 1.5 w/v % HAMA+0.5 w/v % alginate, and 0.5 w/v % HAMA+2.0 w/v % GelMA. All hydrogels were incubated in 2.0 w/v % FITC-Q. chitosan solution in PBS until fully shrunken and then rinsed twice with PBS. The hydrogels were weighed and measured (diameters and heights), and then incubated at 37° C. in individual vials containing 1 mL of PBS per vial. The PBS was completely changed every 2 day. The absorbance (k=488 nm) of the supernatant was determined on Days 1, 3, 7, 11, 15, and 21. In addition, the diameters and heights of the hydrogels were also measured at these time points. After all measurements, the hydrogels were placed back into the vials and fresh PBS was added. By measuring the absorbance of a standard curve of known FITC concentrations at 488 nm and through factoring in the degree of FITC labeling of the Q. chitosan, the amounts of FITC-labeled Q. chitosan released (μg mL⁻¹) from the hydrogels per time point were calculated.

SEM sample preparation and imaging. Hydrogels consisting of 1.0 w/v % HAMA were fabricated and incubated in 2.0 w/v % HMw chitosan in 1.0 v/v % acetic acid aqueous solution for 24 h and freeze-dried, or freeze-dried immediately post-fabrication. The freeze-dried samples were cut using a razor blade, and subsequently sputter-coated with a nanometer-layer of Pt. The sputter-coated samples were imaged with SEM (Phenom™, FEI, The Netherlands). An electron beam of 5 kV was used, and the samples were imaged at 1000 times of magnification.

Extrusion printing. Constructs were first designed by 3D Studio Max (Autodesk, USA) and sliced by Repetier (Hot-World, Germany). An Allevi 2 bioprinter (Allevi, USA) was used to fabricate the constructs. For the extrusion printing of hexagonal patterns, the printhead moving speed was 6 mms⁻¹, and the inks were crosslinked by exposing to UV light (˜10 mWcm⁻², 360-480 nm, 40 s). In this case, we did not use in situ photocrosslinking for our printing processes but performed post-printing photocrosslinking. The HAMA inks were sufficiently viscous to maintain the shape stability immediately post-printing. The printed and photocrosslinked HAMA hexagons were immersed in the chitosan solutions (0.5, 1.0, 1.5, 2.0, 3.0 or 5.0 w/v % in 1.0 v/v % acetic acid aqueous solution) for 24 h. We used microscopy (Eclipse Ti, Nikon, Japan) and a camera (Canon, Japan) to image the constructs after 2 h and 24 h of incubation. We also measured the printability and the Young's modulus of HAMA constructs made with different HAMA concentrations before and after shrinkage.

The alginate and chitosan hexagons were printed in the same way, where alginate hexagons were crosslinked in 0.3-M CaCl₂ and chitosan hexagons crosslinked with glutaraldehyde (800 μM).

For the pyramid (six 10-mm lines converging in four vertices) hydrogel fabrication, the construct was designed and sliced by the same software as mentioned above. The ink consisted of 2.0 w/v % HAMA, 0.1 w/v % Irgacure 2959, and fluorescent microbeads (purple, 15-35 μm; CREATEX, USA), where a 1.5 w/v % gelatin type A (Sigma-Aldrich) hydrogel bath (formed by cooling at 4° C. for 40 min) was used as the support matrix to facilitate freeform printing of the pyramid. The structure was printed by an Allevi 2 bioprinter equipped with a 23 G needle (BD Biosciences, USA), followed by crosslinking by exposing to UV light (˜10 mW cm⁻², 360-480 nm) for 60 s. After photocrosslinking, the gelatin bath was heated and stepwise replaced with water. Finally, the 3D-printed pyramidal structure was incubated in 1.0 w/v % HMw chitosan in 1.0 v/v % acetic acid aqueous solution for 24 h for complete shrinkage.

Sacrificial printing. Sacrificial printing based on Pluronic F127 followed previously established protocols. Kolesky et al., Proc. Natl Acad. Sci. USA 113, 3179-3184 (2016). Pluronic F127 (Sigma-Aldrich) solution was used as the fugitive ink in sacrificial printing, which is a hydrogel at room temperature but liquefies at low temperatures. Specifically, 40 w/v % Pluronic F127 aqueous solution was used for printing the fugitive templates. A PDMS mold (length: 1.5 cm, width: 0.5 cm) was first made. The 2.0 w/v % HAMA solution was cast into the PDMS mold at a thickness of 0.2 cm and UV-crosslinked (10 mW cm⁻², 360-480 nm, 40 s) to act as the base layer. Then, Pluronic F127 (40.0 w/v %) mixed with fluorescent microbeads (red) was printed onto the HAMA gel surface. Subsequently, another layer of 2.0 w/v % HAMA solution was poured into the mold to cover the Pluronic, immediately followed by another UV crosslinking procedure. The construct was placed in water overnight at 4° C. to liquefy and remove the Pluronic, leaving the open channel. Later, the block with open channel was immersed in a HMw chitosan solution (2.0 w/v % in 1.0 v/v % acetic acid aqueous solution) for 24 h. The diameter change of the channel was recorded before and after shrinking. Sacrificial printing of GelMA constructs was done in a similar fashion as for HAMA. It should be noted that, the Pluronic fugitive ink was not completely removed and the residual coating on the channels containing the fluorescent microbeads facilitated visualization.

Sacrificial printing in combination with an embedded freeform printing strategy was also demonstrated, where a blend solution of GelMA (2.5 w/v %) and HAMA (0.5 w/v %) containing 0.1 w/v % photoinitiator was cast into a PDMS mold and placed at 4° C. for 30 min until it became a semi-solid hydrogel bath. Then, 5.0 w/v % gelatin type A was prepared as the bioink at room temperature and printed directly into the GelMA/HAMA bath as a serpentine microfiber, then UV-crosslinked (10 mW cm⁻², 360-480 nm, 60 s). The gelatin was subsequently washed out by incubating the block at 37° C. to form the microchannel. To shrink, the blocks with open channels were immersed in the HMw chitosan solution (2.0 w/v % in 1.0 v/v % acetic acid aqueous solution) for 24 h. HUVECs were subsequently seeded into the microchannels post-shrinking. Perfusion of the channels before and after shrinking was also demonstrated.

Alternatively, PCL meshes were fabricated by the MEW technique and used as sacrificial templates and 2.0 w/v % HAMA was cast around the templates. To leach the PCL template from the HAMA constructs, a multi-stage removal process was optimized. The constructs were immersed sequentially in de-ionized water for 1.5 h, 50 v/v % acetone (Sigma-Aldrich) in water for 1.5 h, in 100 v/v % acetone overnight, 50 v/v % dichloromethane (DCM, Sigma-Aldrich) in acetone for 1.5 h, and 100 v/v % DCM overnight. After dissolution of the PCL, the hydrogel was treated in the reverse order of the steps described until in 100% de-ionized water for rehydration. Later, the block with open channels was immersed into the chitosan solution (2.0 w/v % in 1.0 v/v % acetic acid aqueous solution) for 24 h. The diameters of channels and the lengths of grids were recorded and analyzed.

Coaxial printing. A coaxial printhead containing two injection channels was fabricated, where the size of the internal needle was 23 G and the outer was 16 G. These two needles were fixed concentrically with epoxy resin (Devcon, USA). The internal channel was perfused with a 0.3-M CaCl₂ solution (Sigma-Aldrich) and the external channel was used to print blend inks of HAMA (0.5, 1.0, 1.5, 2.0, or 2.5 w/v %)+0.5 w/v % alginate+0.5 w/v % photoinitiator. Both layers of flows were controlled by syringe pumps (NE-1000, New Era Pump Systems Inc, USA) and the extrusion rates were set at 400 μL min⁻¹ (0.3-M CaCl₂ solution) and 200 μL min⁻¹ (inks). The CaCl₂) solution was used for immediate physical crosslinking of the alginate component for tube formation during printing, whereas the tubes were subsequently shrunken in the HMw chitosan solution (2.0 w/v % in 1.0 v/v % acetic acid aqueous solution) for 24 h and UV-crosslinked (˜10 mW cm⁻², 360-480 nm, 40 s). The ID, OD, WT, as well as the ratios of OD/ID and OD/WT before and after shrinkage were all measured. Alternatively, a coaxial printhead made of 30 G/18 G needles was also produced to print smaller-sized tubes using the blend ink containing 1.0 w/v % HAMA or 5.0 w/v % GelMA, together with 0.5 w/v % alginate and 0.5 w/v % photoinitiator.

Visualization of shrinking distortions using non-rigid registration. The distortions of co-registered pre-shrinking/2-h shrinking structures and post-shrinking structures were visualized by deforming the pre-shrinking/2-h shrinking images using a non-rigid registration process to attempt an exact match of the post-shrinking images of the corresponding samples. Using a B-spline based nonregistration algorithm (D.-J. Kroon, “B-spline Grid, Image and Point based Registration”), which generates a deformation grid between the preshrinking/2-h shrinking (green) and post-shrinking (magenta) patterns, we were able to map the deformation between them. Pre-shrinking/2-h shrinking and post-shrinking images were first converted to binary images using the Matlab function im2bw (Matlab Documentation), which converts an image to a binary image, based on threshold, by replacing all the pixels in the input image with luminance greater than the level 1 (white) and replacing all other pixels with the value 0 (black). Both images were smoothened for faster registration using a Gaussian blur filter, with a standard deviation of 5 pixels.

Shrinking bioprinting in the presence of cells. MCF-7 breast cancer cells (American type culture collection [ATCC], USA) were suspended in 2.5 w/v % GelMA+0.5 w/v % HAMA aqueous solutions at a density of 1.0×10⁷ cells/mL, 0.3 w/v % photoinitiator (lithium phenyl-2,4,6-trimethylbenzoylphosphinate, LAP; Allevi) was added for inducing photocrosslinking (˜10 mW cm⁻², 360-480 nm, 20 s). Half of the samples were shrunken once for 4 h (single shrinkage, the 1st day) in 1.0 w/v % Q. chitosan in DMEM supplemented with 10 v/v % fetal bovine serum (FBS, Gibco), or shrunken twice at 2 h each (successive shrinkage, the 1st day and the 3rd day). In the single shrinkage group, live/dead staining was performed before and after shrinking on the 1st day, the 3rd day, and the 5th day. For the successive shrinkage group, the live/dead staining was carried out before shrinkage and subsequently on the 1st day, the 3rd day, and the 5th day. The specimens with cells were rinsed with PBS and incubated with 2 μM of calcein-AM and 4 μM of ethidium homodimer-1 (Invitrogen, USA) for 30 min to examine viability. Moreover, the cells were also stained for Ki67 as a proliferation marker. The samples were fixed in 4% paraformaldehyde (Thermo Fisher, USA) for 15 min, permeabilized with 0.05% Triton X-100, and then blocked with 2% FBS and 2% bovine serum albumin (BSA, Sigma-Aldrich) in PBS. Samples were incubated with recombinant anti-Ki67 antibody conjugated to Alexa Fluor® 594 (Abcam, USA) overnight at 4° C. FITC-phalloidin (Cytoskeleton, USA) was used to stain for Factin and the nuclei were counter-stained with 4′,6-diamidino-2-phenylindole (DAPI, Vector Laboratories, USA). The samples were then rinsed in PBS and visualized using confocal laser scanning microscopy (LSM880, Zeiss, Germany) and measurements were made using image J. The same protocols were used for shrinking HUVECs (ATCC)-encapsulated hydrogel constructs and associated analyses, only the cultures were maintained in endothelial cell growth medium (EGM-2, PromoCell, USA) and the constructs were shrunken in 1.0 w/v % Q. chitosan in EGM-2. C2C12 mouse skeletal myoblasts (ATCC) were also encapsulated in the same GelMA/HAMA hydrogel and evaluated against the shrinking process. The samples were fixed in 4% paraformaldehyde for 15 min, permeabilized with 0.05% Triton X-100, and then blocked with 2% FBS and 2% bovine serum albumin in PBS. FITC-phalloidin was used to stain F-actin and the nuclei were counter-stained with DAPI. The samples were visualized by an inverted fluorescence microscope (Eclipse Ti).

For the IC₅₀ experiments, MCF-7 cells and HUVECs were cultured in 96-well plates until confluency. The cells were exposed to different concentrations (0.5 μg mL-1-0.5 mg mL⁻¹) of Q. chitosan dissolved in culture medium for various lengths of time (30 min-24 h). After exposure, the cells were incubated with the PrestoBlue™ Cell Viability Reagent (Thermo Fisher) in medium for 30 min and measured for absorbance.

We further tested the shrinkage of sacrificially bioprinted GelMA/HAMA constructs in which GFP-HUVECs (Angio-Proteomie, USA) were encapsulated within the hydrogels where each construct contained a central microchannel formed by removing the Pluronic template embedded with red fluorescent microbeads. The procedure was similar to that without cells, but the Pluronic was washed out in PBS after liquefying at 4° C. for 5 min. Before and after shrinking in 1.0 w/v % Q. chitosan in EGM-2. The samples were visualized with the inverted fluorescence microscope (Eclipse Ti).

### Example 2: Resolution-Enhancements for Hydrogels Fabricated with Light-Based 3D Printing Through Complex Coacervation

An important aspect of 3D printing techniques is the attainable spatial resolution of a printed object. Typically, the 3D printing resolution is increased through improving the printer hardware and/or software. In a recent work, an alternative method to improve the resolution was established by shrinking the hydrogels post-printing. Gong et al., Nature Communications 11(1), 1-14 (2020). This method is based on the incubation of a printed hydrogel based on a charged polymer network in an aqueous solution containing oppositely charged polymers. The resulting electrostatically driven complexation of the free polyelectrolytes into the oppositely charged matrix causes water expulsion from the gel and results in significant shrinking of the hydrogel construct (FIG. 6A). Specifically, we have previously shown that reductions in volume down to 10-20% of the original construct were attainable without changing the printer hardware/software.

The process of forced water expulsion from a gel is often referred to as ‘syneresis’. Syneresis is the result of an event that drastically increases the hydrophobicity of the initially hydrophilic hydrogel network. G. W. Scherer, Journal of Non-Crystalline Solids 108(1), 18-27 (1989). This reduction in water retention ability allows for water expulsion and simultaneous gel shrinking. The mechanism driving the expulsion of water from the hydrogels in this method is known as complex coacervation. Typically, complex coacervation refers to the liquid-liquid phase separation that occurs when two oppositely charged soluble polyelectrolytes of sufficient strength electrostatically bind, release their counterions, and form complexes. De Kruif et al., Current opinion in Colloid and Interface Science, 9(5), 340-349 (2004). These complexes expel water and become denser, causing a macroscopically phase-separated polymer-rich bottom phase and a water-rich polymer-poor top phase. Recent works have found that complex coacervation can also occur when one of the polyelectrolytes is crosslinked into a network. Schuurmans et al., Soft matter 14(30), 6327-6341 (2018). In this case, when a charged hydrogel network is infiltrated by the oppositely charged polyelectrolyte, complexation through electrostatic binding and hydrogel dehydration through water expulsion from the gel consequently occur.

Previously, hydrogel shrinking through complex coacervation was shown to have the potential for use in traditional additive manufacturing techniques for hydrogel fabrication such as extrusion-based printing. Gong et al., Ibid. The developed post-processing method has however, not yet been applied to light-based 3D printing techniques. In recent works, printed structurally sophisticated hydrogels have been made in fabrication times as low as 30 seconds, using a new technique termed ‘volumetric printing (or computed axial lithography)’. Cook et al., Adv Mater, 32(47): e2003376 (2020). This technique allows the formation of hydrogel shapes that would be otherwise difficult to form using traditional additive manufacturing techniques. Additionally, cell-seeded hydrogel structures resembling miniaturized trabecular bone have also been successfully volumetrically printed and cultured. Volumetric printing utilizes the controlled exposure of a series of 2D patterned laser beams to a rotating vat of photopolymerizable resin. Loterie et al., Nature communications 11(1), 1-6 (2020). When all these different light patterns combine, reaching a critical dose distribution, it allows polymerization of the resin, resulting in selective crosslinking. This selective crosslinking leads to instantaneous formation of the desired object within the vat. Upon removal of the object from the vat, it can be processed and used. The main benefits of volumetric printing over existing layer-by-layer photopolymerization-based techniques (e.g. stereolithography or digital light processing) are drastically reduced object generation times, lower risk of unwanted product anisotropy, and the ability to process viscous solutions or gels.

In this study, a detailed investigation of the parameters affecting hydrogel shrinking speed and shrinking extent is reported. These insights enable, for the first time, tunable post-processing of printed hydrogels, making the process simpler, more time-efficient, and more attractive as a general post-printing method. Here, the mechanisms of complexation-induced hydrogel shrinking are also elucidated to allow tunable post-processing. As a unique example of this generally applicable method, volumetrically printed hydrogel structures are fabricated with greatly increased printing resolution, so as demonstrations with other light-based printing methods such as digital light processing (DLP).

### Results and Discussion

### Hydrogel Shrinking Capacity

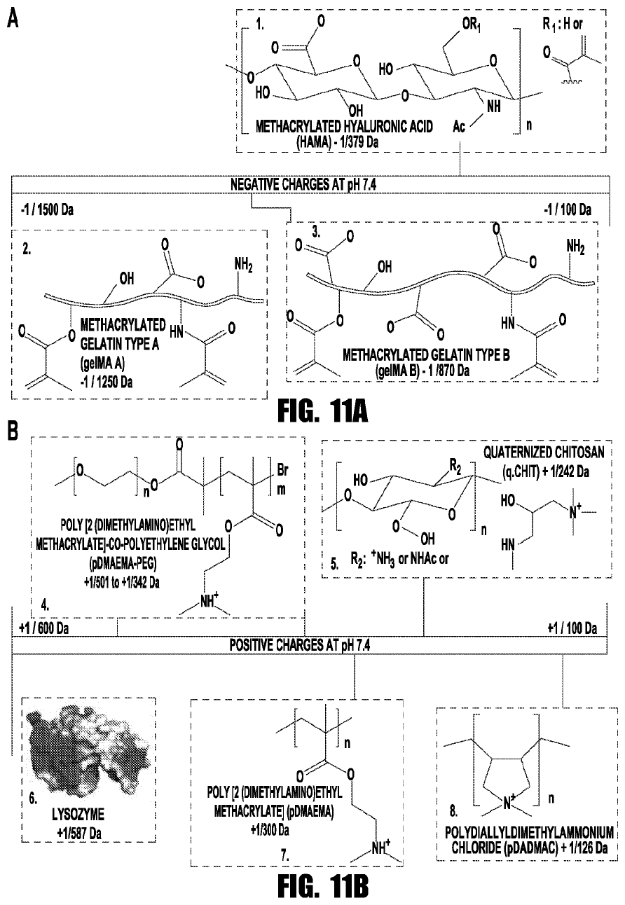

To determine what governs the capacity of a hydrogel to shrink, the effects of parameters such as the polymer charge density and initial hydrogel macromer concentration were systematically investigated. To do this, a series of anionic polymers with varying molecular weights and charge densities were derivatized with methacrylic anhydride to yield photopolymerizable macromers. For all polymers, macromers with different degrees of methacrylation (DM) were formed. The derivatization protocols and polymer specifics are described in the methods section (FIG. 11A). First, two types of gelatin were modified to yield gelatin methacryloyl (gelMA) type A and type B. Gelatin type B has a higher charge density than type A due to differences in processing and the initial collagen source. In addition, hyaluronic acid was derivatized to form hyaluronic acid methacrylate (HAMA). The order of the negative charge densities of these polymers is gelMA A<gelMA B<HAMA. Similarly, a library of polycations with varying charge densities and average molecular weights were used. Individual polymer molecular structures and characteristics are further illustrated in FIG. 11B. The positive charge densities of these polycations from low to high are lysozyme<pDMAEMA-PEG<pDMAEMA<quaternized chitosan<pDADMAC.

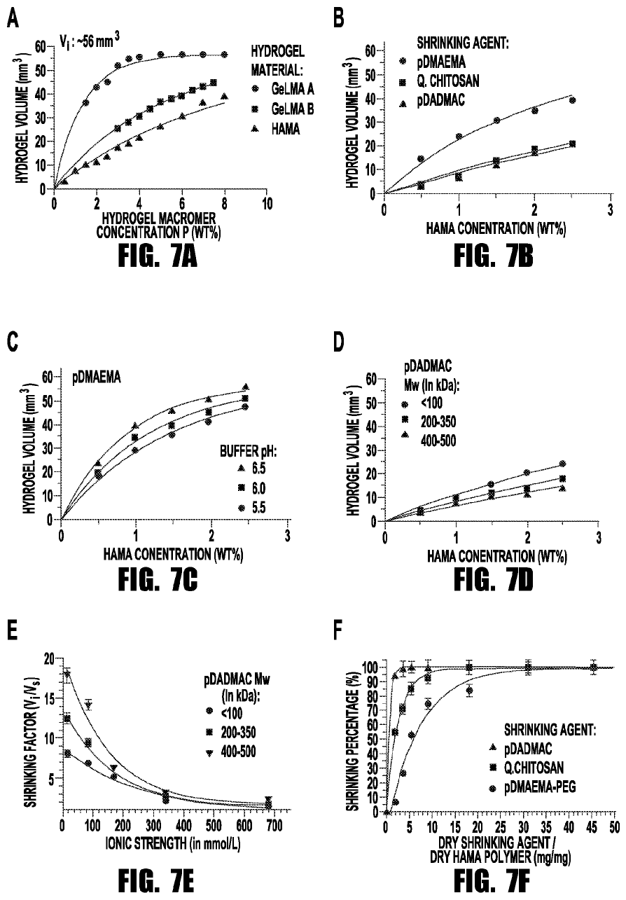

After photopolymerization in the presence of a photoinitiator, hydrogel discs (with initial volume, Vi=56 mm³) were incubated with different aqueous polycation solutions in a variety of conditions. After their full shrinking potentials were reached, the hydrogel dimensions were measured, and their shrunk volumes (Vs) were determined. The first observation from this set of experiments was that the hydrogel shrinking capacity was highly dependent on the initial hydrogel macromer concentration (P) (FIG. 7A-D). All hydrogel-polycation combinations showed that a higher-wt % hydrogel would shrink less as compared to a hydrogel made with a lower initial macromer wt % subjected to the same conditions. In the studied post-processing method, the complexation of the polycation to the network acts as the syneresis trigger that makes the hydrogel more hydrophobic and causes water expulsion. By increasing the initial hydrophilic macromer content in a hydrogel of a fixed volume, more hydrophilic polymer is incorporated into the hydrogel. This increased concentration of hydrophilic polymer increases the relative water retention capacity of the hydrogel. The increased retention capacity allows the gel to remain more hydrophilic after complexation and therefore retain more water.

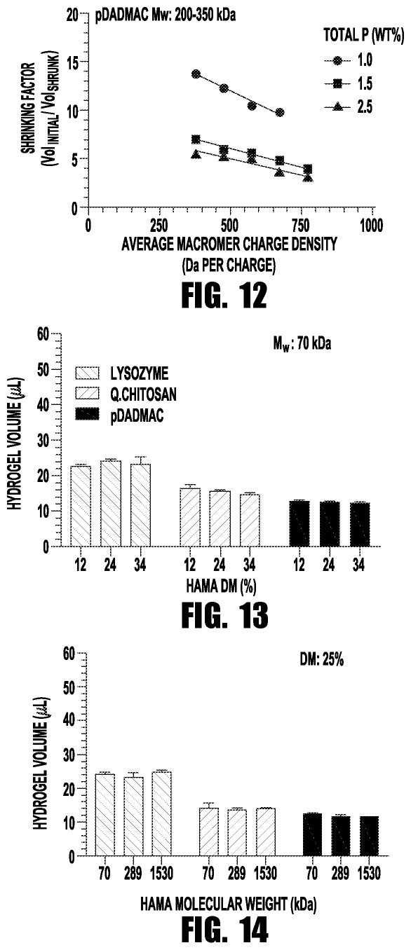

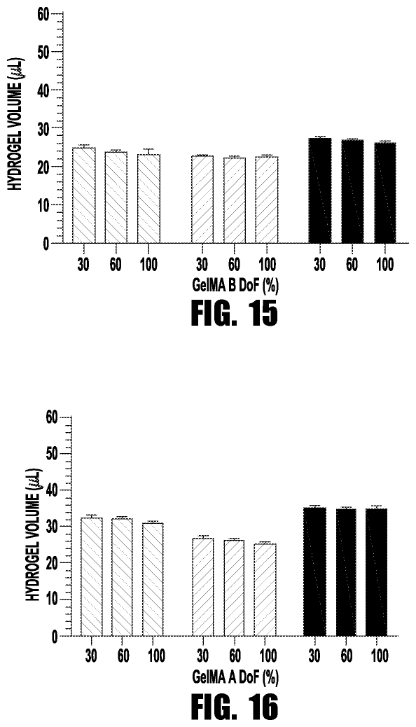

Factors that increase the strength of the electrostatic interaction between the hydrogel network and the polycations were expected to lead to more extensive hydrogel dehydration. To test this, parameters affecting the strength of the electrostatic interactions between the anionic hydrogel network with the soluble polycations were systematically investigated. First, the effects of changing the network anionic charge density were studied. Hydrogels made with macromers of higher negative charge densities were found to experience more dehydration when incubated with polycations than those comprising of macromers of lower negative charge densities (FIG. 7A). For example, hydrogels made from weakly negatively charged gelMA A only showed significant shrinking below 4 wt % of the initial gelMA A content. The dependence of shrinking on macromer charge density was further confirmed by following the shrinking capacities of multicomponent hydrogels made from combinations of two anionic macromers, leading to varying negative charge densities (FIG. 12). Specifically, it was found that increasing the total hydrogel charge density of a range of gelMA B/HAMA combinations resulted in a linear decrease in the hydrogel shrinking factor (Vᵢ/Vₛ). Polycation charge density was also found to influence hydrogel shrinking capacity, as generally, hydrogels incubated with polycations of higher charge densities but similar M_{w} were found to shrink more (FIG. 7B and FIGS. 14-16).

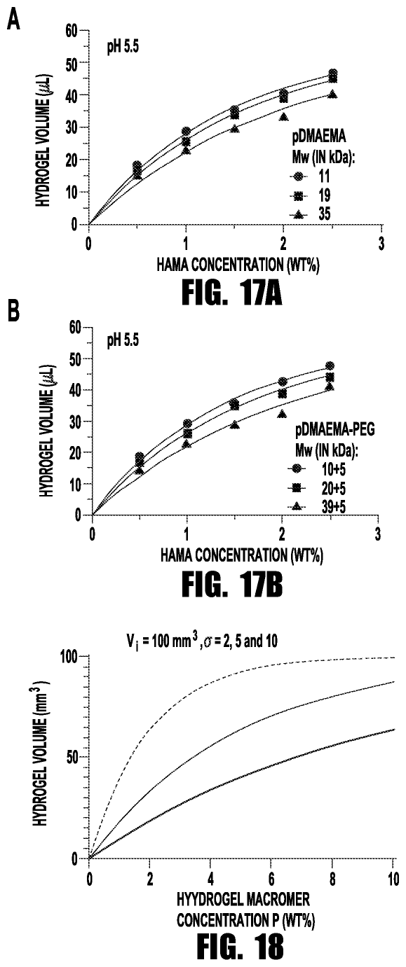

Hydrogels incubated with lysozyme in isotonic conditions (170 mM of ionic strength and pH 7.4) did not shrink at all. Only at the reduced ionic strength (85 mM, pH 7.4), hydrogel shrinking was observed for lysozyme (FIGS. 13-15). This result suggested that lysozyme is not sufficiently cationic to induce gel dehydration under physiological conditions. Indeed, previous studies done on shrinking of HAMA microgels with lysozyme as well as interaction studies of free HA with lysozyme in various ionic strengths reported as well that the required complex coacervation between HAMA/HA and lysozyme only occurs at lower ionic strengths. Water et al., European journal of Pharmaceutics and Biopharmaceutics 88(2), 325-331 (2014). When HAMA hydrogels were incubated in more positively charged polycation solutions, such as those containing pDMAEMA-PEG and pDMAEMA, the resulting complexation was strong enough to elicit hydrogel dehydration in physiological conditions (FIG. 7B and FIG. 17). Similarly, incubation of HAMA hydrogels in polycations with even higher charge densities than pDMAEMA such as Q(uaternized) Chitosan and pDADMAC in comparable conditions led to higher degrees of hydrogel dehydration than observed for pDMAEMA-shrunken hydrogels (FIG. 7B). Although the block-copolymerization of pDMAEMA with PEG reduces the average charge density significantly, it does not reduce the pDMAEMA block charge density. Pegylation is shown not to effect hydrogel dehydration. This dataset indicated that the electrostatic interaction between the hydrogel network and the polycations is dependent on pDMAEMA block length and charge density and is not hindered by the PEG block. Reports showing that the binding affinity of pDMAEMA(-PEG) to polyanions such as DNA or heparin is dependent on the pDMAEMA (block) length and is independent of PEG block length, further confirm our observation regarding pDMAEMA block length. Valimaki et al., Biomacromolecules 17(9), 2891-2900 (2016). Importantly, changing the charge density of both the hydrogel network and the polycation can be used to tune hydrogel shrinking.

PDMAEMA was used to study the effect of buffer pH on hydrogel shrinking (FIG. 7C). HAMA hydrogels that were incubated with pDMAEMA in pH 5.5 were subject to a higher degree of dehydration than those incubated at pH 6.0 and 6.5. The investigated pH range hardly affected the charge density of HAMA, as the pKa of its carboxylic acid is estimated to be approximately 3-4. This increased dehydration therefore, is again a result of stronger binding of the polycationic pDMAEMA to the negatively charged hydrogel matrix. Logically, this increased interaction between pDMAEMA and the HAMA network is a direct effect of the increase in positive charge density at lower pH values of the pDMAEMA used, as the pKa of this polymer is 7.5. The influence of pH on polycation charge density can also be used to precisely tune the size of the printed hydrogels to achieve desired resolutions.

Next, the effect of the average polymer M_{w} on hydrogel shrinking capacity was studied. To do this, hydrogels were shrunken with three different polycations of three different average M_{w}'s (FIG. 7D and FIG. 17A, B). All combinations of polycations and hydrogels studied showed that incubation of a hydrogel in a higher average M_{w} polycation-based solution would lead to significantly more dehydration and shrinkage. This observation corresponds with previous studies on complex coacervation between oppositely charged polymer solutes, where increasing the M_{w} of either polymer was found to lead to increased strength of complexation. De Kruif et al., Current opinion in Colloid and Interface Science 9(5), 340-349 (2004). In contrast to what is observed for complexation between polymeric solutes, it was found in our case that increasing the M_{w} of the anionic macromers used to produce the hydrogels did not have any effect on hydrogel dehydration. This lack of effect is likely due to the crosslinking step performed during hydrogel fabrication, which links all macromers together into a large anionic network and cancels out any potential effect of macromer Mw. Similarly, no effect of changing the macromer methacrylation or methacryloyl substitution degree (and thus the network crosslinking density) on hydrogel dehydration capacity was observed (FIGS. 13, 15, 16). Summarizing, tuning the level of hydrogel shrinking can be done by altering the M_{w} of the polycations used.

Lastly, the effect of ionic strength on hydrogel-polycation interaction and hydrogel shrinking for several M_{w}'s of pDADMAC was studied (FIG. 7E). As the solution ionic strength was increased from 17 to 680 mM, the shrinking capacity of the studied HAMA hydrogel was found to exponentially decrease. As expected, differences between the various pDADMAC polymers could be observed, as shrinking induced by higher-average M_{w} polycations led to significantly higher shrinking capacities, especially at lower ionic strengths. This dependency of hydrogel shrinking (and thus strength of complexation) on ionic strength is due to the increased screening of charges by ionic solutes that occurs at higher ionic strengths. Hariharan et al., Macromolecules 31(21). 7514-7518 (1998). Screening of the charges present on both the polycation and hydrogel network leads to weaker electrostatic polymer-hydrogel interaction and results in reduced water expulsion from the hydrogel.

From the above-described experimental observations, two major parameters were identified to affect hydrogel shrinking capacity: the initial macromer concentration used to create the hydrogel network and the strength of (electrostatic) interaction between the polycation and anionic network. The latter of these factors is determined by several parameters such as the charge densities of the polycation and the anionic network, the buffer conditions (i.e., pH and ionic strength), and the M_{w} of the polycation. It can be expected that other non-electrostatic interactions influence this interaction strength as well. To quantify and compare the shrinking capacities of polycation-hydrogel combinations under specific conditions, the following equation to describe maximum hydrogel shrinking capacity was derived:

Vₛ−Vᵢ(1−e^{-P/χ}) Eq. 1

Where χ is the polymer-network interaction parameter, and the initial macromer concentration is represented as P. Vᵢ and Vₛ are the initial and shrunken hydrogel volumes, respectively. It should be noted that at a fixed P, a higher χ results in a smaller hydrogel shrunken volume (Vₛ) and thus a higher water expulsion. See FIG. 18 for an explanatory graph where several theoretical y values are plotted. To test the veracity of Eq. 1, it was fitted to the data shown in FIG. 7A-D and FIG. 17A, B (solid and dashed lines show interpolated and extrapolated best-fits, respectively). All data were found to fit very well (typical R²>0.98) to the equation, suggesting that the maximum hydrogel shrinking capacity is indeed only dependent on initial macromer concentration and the strength of polymer-network interaction. By using this equation and deducing the k factor, we can quantify the strength of interaction between polycation-network pairs and predict the hydrogel shrinking behavior for a hydrogel of any volume, allowing predictable post-processing of printed hydrogels.

Although the main parameters influencing the strength of polymer-network interaction were elucidated, the effects of additional parameters may still be seen when regarding the calculated y factors for HAMA and gelMA B hydrogels shrunken with Q.Chitosan and pDADMAC. For instance, shrinking a HAMA hydrogel with pDADMAC resulted in equally dehydrated hydrogels as compared to incubation with Q.Chitosan of a similar M_{w}. Also, despite the higher charge density of pDADMAC compared to Q.Chitosan, gelMA B-based hydrogels shrank more when incubated with Q.Chitosan than pDADMAC of a similar Mw (FIG. 7B). This is likely due to additional favorable non-electrostatic interactions between Q.Chitosan and gelMA/HAMA networks such as hydrophobic interaction, dipole interactions, Van der Waals force, and hydrogen bonds. These additional interactions can contribute to polymer-network interaction strength, k, and thus increase hydrogel dehydration. Similarly, the amphionic nature of gelMA B could also allow for repulsive polymer-network interactions, leading to lower interaction strength for more positively charged polymers.

To test whether the shrinking of hydrogels can be tuned by regulating the amount of polycation absorbed into the hydrogels, HAMA hydrogels (Vᵢ=56 mm³) were incubated with 0 to 45 mg of pDADMAC, Q.Chitosan, or pDMAEMA-PEG per mg of dry HAMA polymer in a fixed volume. Accordingly, HAMA hydrogels indeed were only partially shrunken upon exposure to smaller amounts of polycation (FIG. 7F). Subsequently, the minimum amount of polycation needed to shrink a hydrogel to its maximum capacity was determined. Specifically, it took ˜2.5-5.4, 18.2, and 31.0 mg of pDADMAC, Q.Chitosan, and pDMAEMA-PEG per mg of HAMA, respectively, to fully shrink the hydrogel (FIG. 7F). Changing the average M_{w} of the pDADMAC used did not influence the amount of pDADMAC necessary to induce full hydrogel shrinking. The amount of polycation necessary to shrink is related to the charge density of the polycation, as the order of charge density of the polycations studied (pDADMAC>Q.Chitosan>pDMAEMA-PEG) is the opposite as the order found for the amounts of polycation necessary to fully shrink the HAMA hydrogel.

### Hydrogel Shrinking Rate

For future applications of our unique post-processing technique, it is important that the parameters influencing the time it takes until a hydrogel is fully shrunken are understood. Being able to control and limit the total time until full hydrogel shrinking is vital for the usability of this technique. Through fluorescence microscopy studies, it was previously shown by us that the HAMA hydrogel shrinking rate was directly related to the penetration of the polycations into the hydrogel. Specifically, it was observed that the polycations could permeate and bind throughout the hydrogel and eventually homogeneously distribute throughout its matrix. Considering that the transport of solutes through hydrogels is generally diffusion-based, we hypothesized that hydrogel shrinking speed is related to the diffusion speed of the polycations into the hydrogel matrix. In general, at constant temperature and pressure, the diffusion coefficient D (in m²/s) of a solute as it travels from an area in which a high concentration of the solute is present to a partition where no solute is present is given by Fick's law:

$${n}_{P} = - D \frac{{dC}_{P}}{dy} , \qquad Eq . 2$$

where dC_{P}/dy indicates the solute molar concentration gradient present between the two studied partitions and n_{P} is the molar diffusion flux of the solute.

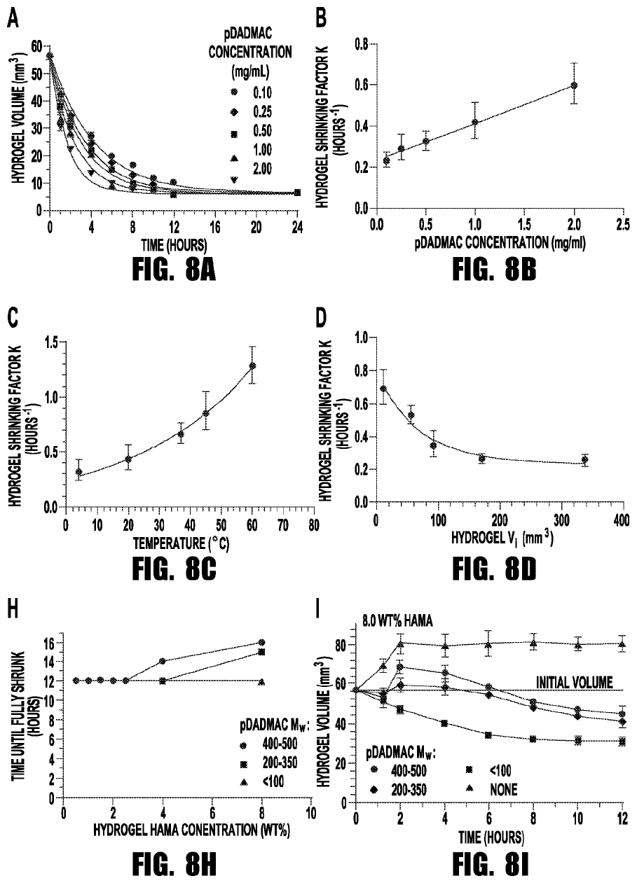

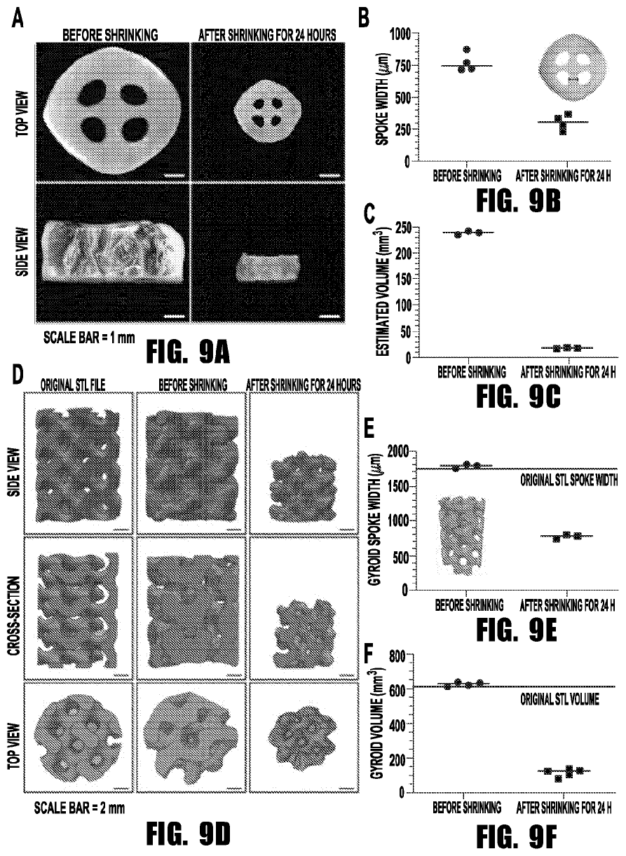

To discern if hydrogel shrinking rates could be controlled through altering the polycation diffusion speed, hydrogels were incubated with polycations in several conditions and their volumes were measured over time (see FIG. 8A for an example). Increasing the concentration of the polycation in the incubation buffer was shown to linearly decrease the time until full hydrogel dehydration (FIG. 8A, B). This linear relation between polycation concentration and hydrogel shrinking is as predicted by Eq. 2. To explain, the polycation molar concentration gradient dCP/dy is directly related to the initial pDADMAC concentration and increasing it will directly increase the molar polycation flux. By increasing this molar flux, the hydrogel dehydration speed also increases linearly.

It could be expected that increasing the buffer temperature would decrease the time exponentially until the full hydrogel shrinking, through an increase of the polycation diffusion coefficient. Indeed, it was observed that by varying the incubation temperature during hydrogel shrinking from 4 to 60° C., it was possible to exponentially reduce the time until full hydrogel shrinking (FIG. 8C).

Hydrogel volume and surface area are important parameters when considering diffusional transport of solutes into hydrated polymeric networks. For the systems studied here, increasing the initial hydrogel volume caused the required time for full hydrogel shrinkage to increase exponentially (FIG. 8C). Again, this relationship followed established hydrogel solute transport mechanisms. Specifically, it can be expected that as the total hydrogel volume increases, the time until total hydrogel shrinking is prolonged exponentially via increasing the polycation diffusion distance throughout the network. Similarly, when the hydrogel surface area (HSA) divided by the initial hydrogel volume was plotted against the time until full hydrogel dehydration, an inverse exponential relation was found (FIG. 19). This relationship corresponds with the earlier defined theoretical models and again provides evidence that polycation diffusion governs hydrogel shrinking speed.

To verify that hydrogel shrinking rate is dependent on the polycation diffusion, the effects of HAMA hydrogel mesh size and pDADMAC hydrodynamic volume effects were investigated (FIG. 7E, F). By generating HAMA hydrogels with initial macromer concentrations of 0.5 to 8.0 wt %, the initial network mesh size could be reduced. Furthermore, by using the three pDADMAC polymers of average M_{w} of <100, 200-350, and 400-500 kDa that were used earlier, several polycation hydrodynamic volumes could be studied. It took 12 h to shrink HAMA hydrogels of ≤2.5 wt %, regardless of pDADMAC type used. It was only at higher wt % that the HAMA hydrogel mesh would temporarily hinder hydrogel shrinking. Further polycation hydrodynamic volume-dependent effects of the initial hydrogel mesh size were observed for the highest wt % HAMA hydrogel tested, where the differently sized pDADMAC molecules took respectively 15 and 16 h to fully penetrate into and shrink the HAMA hydrogels (8.0 wt %). In contrast, hydrogels incubated in the smallest pDADMAC polymer solution showed similar shrinking rates regardless of the initial HAMA wt % used to create the hydrogels. In FIG. 7F, it could be clearly seen that as the 8.0 wt % hydrogel swelled, before the influx of pDADMAC into the gel and the resulting shrinking commenced. Specifically, when considering the swelling, the mesh size of the 8.0 wt % HAMA hydrogel grew, allowing pDADMAC diffusion. The penetration and complexation of polycations into the networks that result in hydrogel shrinking are thus diffusion-related and can be predicted. This insight is highly useful for hydrogel shrinking as a post-processing method for 3D-printed hydrogels.

### Shrinking of Volumetrically Printed Hydrogels

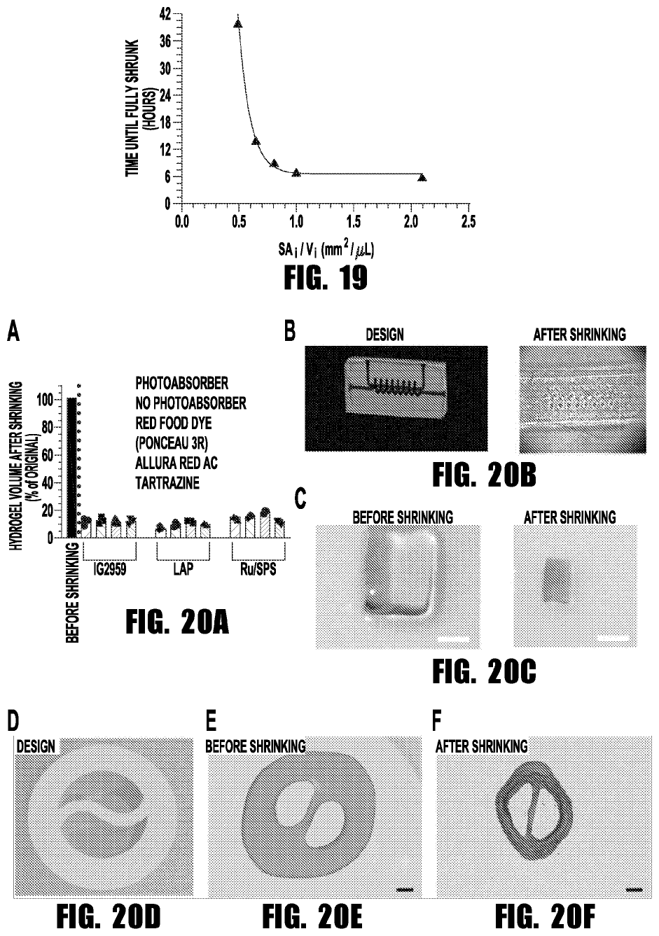

In photopolymerization-based additive manufacturing, successful printing often depends on the use of photoinitiators, photoabsorbers, and photoinhibitors. These additives have become commonplace to increase the printing fidelity. Li et al., Adv Healthc Mater., 9(15): e2000156 (2020). Known photoinitiators such as Irgacure 2959, lithium phenyl-2,4,6-trimethylbenzoylphosphinate (LAP), and ruthenium (II)-sodium persulfate complexes in addition to various photoabsorbers were tested for their potential effects on hydrogel shrinking (FIG. 20A). No discernable effects of any additive on hydrogel shrinking capacity were found, demonstrating the general suitability of the water expulsion technique for printed hydrogels made using various light-based systems.

As an initial test, several DLP-printed structures based on combinations of fish gelMA (used with DLP due to its liquid state at room temperature, instead of mammalian-based gelMA) and HAMA macromers were successfully printed and successfully shrunken (FIG. 20B, C). All initially designed hollow or other features were still present in the shrunken constructs. Next, hydrogel shrinking as a post-processing method for volumetric printing was evaluated. To successfully volumetrically print structures from low-wt % photopolymer solutions, it was necessary to add 0.002 wt % of the radical inhibitor (2,2,6,6-tetramethylpiperidin-1-yl)oxyl (TEMPO). This inhibitor prevented radicals (which were highly mobile in the low-viscosity fluid) from forming off-target crosslinks outside of the volume of interest within the vat and allowed for good print fidelity. Hunold et al., J Phys Chem B., 124(39), 8601-8609 (2020). Initially, a hollow hydrogel disc with a wavy wall insert was printed with a 0.5 wt % HAMA macromer solution (FIG. 21D-F). Interestingly, due to the high amount of shrinking of this printed hydrogel, the resulting gel resembled a crumpled, tortuous wafer-thin structure. The inherent structural weakness of this low-wt % hydrogel caused the gel to lose its shape fidelity over the course of the shrinking process. The partial destruction of the gel indicated that a sufficiently strong hydrogel, able to withstand the stress caused by shrinking should be used for an optimal printing result. Using a 1.0 wt % HAMA macromer solution to volumetrically print and subsequently shrink a perforated disc design proved much more successful (FIG. 9A-C). Shrinking this printed structure resulted in a ˜18× reduction in volume and left only ˜5.6% of its original volume remaining after the water was expulsed (FIG. 9C). Importantly, this 18× reduction in volume corresponded with the values from the data gathered earlier (FIG. 7), where a factor of 17.9±0.9 was found for the same conditions. Furthermore, measurements on the internal spoke widths of the hydrogels showed that the initial width of ˜770±61 μm was reduced by a factor 2.6 to ˜301±50 μm after shrinking (FIG. 9B). This high amount of shrinking in addition to the full conservation of the important design parameters such as the aspect ratio and shape fidelity prove the excellent suitability of this technique to post-process volumetrically printed hydrogels.

To evaluate whether post-processing of printed objects was compatible with hydrogel designs with hollow cavities, a more complex structure was printed using a 4.0 wt % gelMA B solution (FIG. 9D-F). Specifically, a gyroid structure was chosen for this purpose as it contains numerous hollow structures and can be challenging to print using conventional additive manufacturing techniques at this low macromer concentration. Again, the expulsion-based processing technique proved excellently capable of increasing the printing resolution by reducing the initial printed volume whilst retaining the important structural features. Specifically, a factor of 2.3× reduction in dimensional features, or a 5× reduction in volume, was achieved, as measured on the individual spokes of the gyroid (FIGS. 9E and F, respectively).

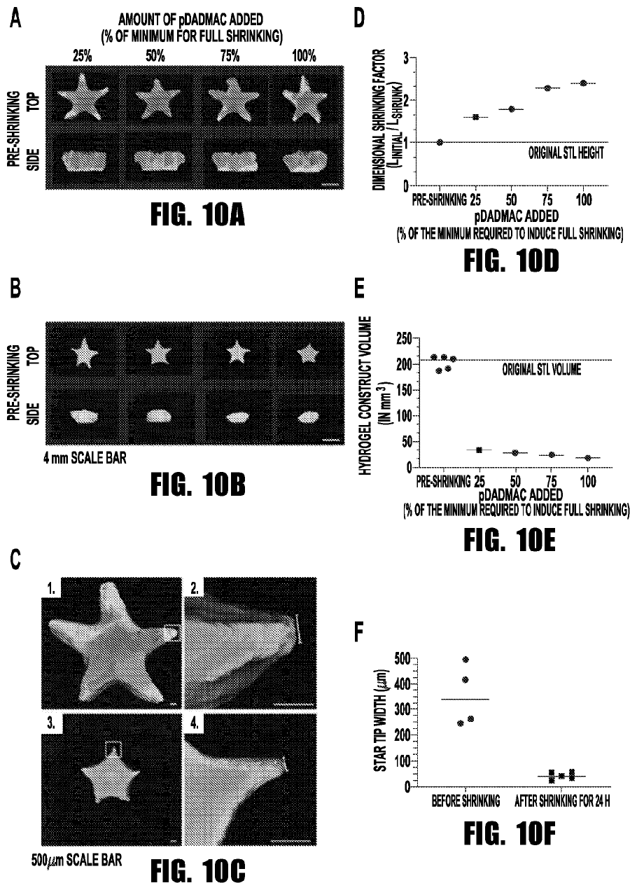

A potential benefit of the water expulsion technique is that the level of water expulsion from a volumetrically printed hydrogel can be controlled, allowing post-printing tuning of the printed hydrogel dimensions. To evaluate if it is indeed possible to tune the dimensions of a volumetrically printed hydrogel, several volumetric prints were made of the same star design (FIG. 10A). These star hydrogels consisted of 1.0 wt % HAMA and were shrunken in physiological conditions (pH 7.4, 170 mM of ionic strength). The polycation used to shrink these gels was pDADMAC (Mw: 400-500 kDa). Each of the stars was incubated with a different total amount of pDADMAC to probe if total printed hydrogel volume was tunable via the amount of polycation added. Theoretically, as the initial design volume of the star was 208 mm³ (or ˜2.1 mg of HAMA by dry weight), it was necessary to incubate it with 0.27, 0.89, 1.83, or >5.62 mg of pDADMAC for it to shrink 25, 50, 75, or 100% of its maximum shrinking capacity (calculated according to data from FIG. 7F). Indeed, upon exposure of the printed constructs to pDADMAC, hydrogel shrinking through water expulsion occurred in a pDADMAC amount-dependent manner (FIG. 10B-D). The shrinking process did not alter the overall shape or fidelity of the printed constructs (FIG. 10B). Furthermore, it could be observed that the dimensional shrinking factors (measured here by dividing the construct height before shrinking by the construct height after shrinking) are 1.6, 1.8, 2.3, and 2.4× for the constructs incubated in 25, 50, 75, and 100% of the required pDADMAC to fully shrink the hydrogel constructs (FIG. 10C). Similarly, the construct volumes after shrinking were 32, 28, 24, and 18 mm³ for stars incubated with 25, 50, 75, and 100% of the required pDADMAC for full shrinking. These results underline the potency of tuning volumetrically printed hydrogel dimensions and volumes through post-processing based on complexation with polycations.

When observing the volumetrically printed star that was shrunken to its maximum capacity (FIG. 10A-C, 100% pDADMAC group), it was clear that the corners of the shrunk star had a width of 42±6 μm. Originally, the width of the tip of the hydrogel star was ˜450 μm. This remarkable reduction in spatial dimensions shows the promise and potential of incorporating post-processing methods into any 3D printing application by shrinking printed hydrogels through water expulsion. To compare, a recent report by Loterie et al. showed that it is possible to resolve a ˜78 μm-wide feature as part of a larger printed acrylic object. Loterie et al., Nature communications 11(1), 1-6 (2020). Several factors influence the printing resolution, among which are the macromer reactivity, free radical inhibition effects, resin viscosity, and optical parameters. For volumetric printing of hydrogels, the typically attained feature sizes are generally in the range of 150-200 μm. Bernal et al., Advanced materials 31(42), e1904209 (2019). Increasing the resolution of volumetric printing with hydrogels will enable smaller features in hydrogel topographies, allowing the fabrication of more complex architectures with numerous potential applications.

### Conclusions

To conclude, hydrogels based on crosslinked anionic polymer networks can be shrunken significantly by electrostatic complexation with polycations in solution. This complexation leads to the formation of a coacervate hydrogel and allows shrinking via the expulsion of water. The hydrogel shrinking capacity was found to be governed by the initial macromer concentration and the strength of interaction between the network and the polycation. This manuscript shows the tunability of the hydrogel shrunk volume through different parameters. An empirical equation fitting the of shrinking capacities for specific systems has been derived. Furthermore, hydrogel shrinking speed was found to be dictated by the diffusion speed of the polycations into the hydrogel matrices and could be controlled by changing diffusion related parameters. Finally, DLP- and volumetrically printed hydrogels were post-processed to yield shrunk hydrogels with similar aspect ratios. Volumetrically printed hydrogels were reduced in volume up to a factor ˜18, or ˜5.6% of their original volume. Importantly, the post-processing method was found to allow shrinking whilst retaining the original features such as hollow structures that can be made with volumetric printing. Using less than the minimum amount necessary to fully shrink a volumetrically printed construct was shown as an easy way to tune hydrogel shrinking extent. Importantly, through optimizing the shrinking parameters, a printed feature size of 42±6 μm was obtained. The insights gained here allow predictable and controlled shrinking of printed hydrogels and significantly drives the resolution of volumetric printing for hydrogels up. In the future, such 3D-printed hydrogels could potentially be used for the development of wearable electronics, biomedical devices, tissue-engineered constructs, and utilized towards various other applications.

### Materials and Methods

### Chemicals

Unless noted otherwise, all chemicals were obtained from Sigma-Aldrich (Zwijndrecht, The Netherlands) and used as received. All solvents were obtained from Biosolve (Valkenswaard, The Netherlands) and were dried (where indicated) using molecular sieves for 24 h prior to use. pDADMAC solutions with average M_{w}'s of <100, 200-350, and 400-500 kDa were purchased from Sigma-Aldrich. Q.Chitosan was purchased from Cool Chemistry (China), pDMAEMA polymers with average M_{w}'s of 11, 19, and 35 kDa were purchased from Polymer Source (Canada) and lysozyme, extracted from chicken egg white (BioUltra grade, >98% pure) was purchased from Sigma-Aldrich.

### Polymer Derivatization, Synthesis, and Characterizations

Methacrylation of Fish gelMA, gelMA Types A and B, and HAMA

All gelatins were derivatized with methacrylic acid to yield gelMA. Van den Bulcke et al. Biomacromolecules 1(1), 31-38 (2000). Specifically, fish gelatin (from cold water fish skin, bioreagent grade), gelatin type B (from bovine skin, 225 g Bloom), and two types of gelatin type A (gel strength ˜175 and 300 g Bloom, respectively, from porcine skin) were all purchased from Sigma-Aldrich (USA) and used as received. Hyaluronic acid of various M_{w}'s (Lifecore Biomedical, USA) was derivatized to yield HAMA using addition of methacrylic anhydride to a HA in H₂O/DMF solution according to our previous protocol. Gong et al., Nature Communications 11(1), 1-14 (2020).

Synthesis of pDMAEMA-PEG Diblock Copolymers

mPEG-pDMAEMA block co-polymers were synthesized using a macroinitiator strategy. The (PEG)₂-ABCPA macroinitiator composed of polyethylene glycol (PEG) chains (M_{w}: 5 kDa) and 4,4′-azobis(4-cyanopentanoic acid) (ABCPA) as azoinitiator for free radical polymerization, was synthetized as described previously. Bagheri et al., Langmuir 34(50), 15495-15506 (2018). Subsequently, mPEG-pDMAEMA (PD) diblock copolymers bearing various block lengths of pDMAEMA (D), were synthesized slightly modifying the method reported by Fliervoet et al. Fliervoet et al., Macromolecules 50(21), 8390-8397 (2017). To remove the inhibitor, 2-(dimethylamino)ethyl methacrylate (DMAEMA) was passed through a column of alumina before use. The (PEG)₂-ABCPA macroinitiator (1 eq.) and various amounts of the DMAEMA monomer (from 64 to 256 eq.) were dissolved in dry DMF in an airtight Schlenk flask, keeping monomer concentration constant (300 mg/mL). To degas the solution, three freeze-pump-thaw cycles were applied, after which the reaction mixture was placed in an oil bath at 70° C. and stirred for 24 hours under the nitrogen atmosphere. The polymer solution was transferred into a dialysis bag (M_{w} cut-off: 14 kDa) and dialyzed against water for 2 days at 4° C. The final PD polymers were recovered by freeze drying and the crude products were obtained as a white powder with a yield of 80-92%. The synthesized polymers were characterized by ¹H NMR spectroscopy and GPC. ¹H NMR spectroscopy was performed on an Agilent 400 MR NMR spectrometer (Agilent Technologies, USA). Chemical shifts are referred to the residual solvent peak (6=7.26 ppm for CDCl₃). Data analysis was performed using MestReNova Software version 12.0.4-22023. The synthesized products were further characterized by GPC using a Waters Alliance System (Waters Corporation, USA) equipped with a refractive index (RI) detector and a PLgel 5 μm MIXED-D column (Polymer Laboratories) using dimethylformamide containing 10-mM LiCl as eluent. The column temperature was set to 65° C. and the flow rate to 1.0 mL/min. Calibrations were performed using PEG (P) standards of narrow and defined molecular weights. All data analyses were performed using Empower 3 Software 2010. For the results regarding polymer characterization, see Table 1.

TABLE 1
Characteristics of mPEG-pDMAEMA (PD) diblock copolymers synthesized by free radical
polymerization. The abbreviations of the polymers follow the composition of the final block

| | Feed initiator:monomer | Mₙ P block | Mₙ D block | Total Mₙ | Total Mₙ | Polydispersity |
|---|---|---|---|---|---|---|
| Polymer | ratio (mol/mol) | (kDa)^{a} | (kDa)^{a} | (kDa)ª | (kDa)^{b} | index^{b} |
| P₅D₁₅ | 1:64 | 5 | 10 | 15 | 12 | 2.0 |
| P₅D₂₀ | 1:128 | 5 | 20 | 25 | 18 | 2.2 |
| P₅D₄₇ | 1:256 | 5 | 39 | 44 | 27 | 2.3 |

ªDetermined by ¹H-NMR
^{b}Determined by GPC

### Hydrogel Shrinking and Swelling Studies

Hydrogels were made from gelMA A or B, fish GelMA, and/or HAMA and were prepared at different concentrations. GelMA formulations were dissolved whilst stirring in MilliQ water at 37° C. for 2 h. HAMA formulations were dissolved into MilliQ water overnight under agitation. Both HAMA and gelMA solutions were supplemented with 0.1 wt % Irgacure 2959 (Sigma-Aldrich) prior to UV crosslinking. The prepared polymer solutions were cast in circular PDMS molds (diameter: 4.5 cm) and subsequently UV-irradiated (methacrylate conversion was determined to be >95%). Subsequently, a biopsy punch (Integra Miltex, various diameters) was used to cut cylindrical hydrogels of 2 mm in height. The hydrogels used for standard measurements (all measurements except the hydrogel volume effect measurements) were 6 mm in diameter and 2 mm in height and all had an initial volume of ˜56 mm³.

Shrinking agents were dissolved overnight in either phosphate-buffered saline (170 mM of ionic strength, pH 7.4) or diluted or NaCl-supplemented phosphate buffer (17-700 mM ionic strengths, pH 7.4) for up to 2.0 wt %. Cut hydrogels were initially measured using Vernier calipers (standard error=30 μm) to determine their volumes and subsequently incubated in the various shrinking agent solutions (volume: 2 mL, n=3 for every condition). Unless stated otherwise, incubation was done at room temperature. Hydrogel diameter and height were monitored either over time to determine hydrogel shrinking kinetics or after full dehydration had occurred to determine maximum shrinking capacity (typically after 12 h).

### DLP Printing

DLP printing was performed using a custom-developed DLP printer. It was built with a commercial DLP projector (ViewSonic PA503W) featuring 3600 lumens brightness of visible light. Specifically, the printing solution contained 0.5 wt % HAMA (with a M_{w} of 1530), 2.0 wt % fish gelMA, 1-mM ruthenium (Ru)/10-mM sodium persulfate (SPS) (Advanced Biomatrix, USA) as the photoinitiator, and Ponceau R (Sigma-Aldrich) as the photoabsorber. Each designed 3D model was sliced into a set of images that were irradiated sequentially onto the resin tank; the printed structure was achieved layer by layer with the speed of approximately 10 μm/s.

### Volumetric Printing

Volumetric printing was done using a volumetric printer (Readily3D, Lausanne Switzerland). Specifically, a 0.5 or 1.0 wt % HAMA or 4.0 wt % gelMA B solution, supplemented with 0.1 wt % LAP was exposed to a light dose of 625 mJ/cm² (exposure time=79.2 s; laser intensity=7.89 mW/cm²) and samples were subsequently washed in PBS to remove un-crosslinked resin. For HAMA based solutions, 0.002 wt % (2,2,6,6-Tetramethylpiperidin-1-yl)oxyl was added. Washed samples were either stained with Alcian Blue for 2 minutes to help microscopic imaging or used without further staining.

DLP and volumetrically printed hydrogels were subsequently imaged and measured using an optical microscope and shrunk by incubation after as described herein. After shrinking, the printed shrunk hydrogels were again imaged and measured via optical microscopy. Volumetrically printed gels were also analyzed using a MicroCT device.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of shrinking a hydrogel, comprising contacting a polyionic hydrogel with a polyionic solution including an ionic polymer having a net opposite charge from that of the polyionic hydrogel for an amount of time sufficient to decrease the volume of the polyionic hydrogel.

2. The method of claim 1, wherein the polyionic hydrogel is applied to a surface using a 3D printing method before contacting the polyionic hydrogel with the polyionic solution.

3. The method of claim 1, wherein the hydrogel is a polyanionic hydrogel, and the polyionic solution is a polycationic solution.

4. The method of claim 1, wherein the hydrogel is selected from the group consisting of hyaluronic acid methacrylate, gelatin methacryloyl, and alginate.

5. The method of claim 1, wherein the ionic polymer is selected from the group consisting of chitosan, quaternized chitosan, lysozyme, poly[2(dimethylamino)ethylmethacrylate], poly [2(dimethylamino)ethylmethacrylate]-co-polyethylene glycol, and polydiallyldimethylammonium chloride solutions.

6. The method of claim 1, wherein the method results in at least a 5 fold decrease in the volume of the polyionic hydrogel.

7. The method of claim 1, wherein shrinking of the hydrogel is complete within 24 hours.

8. The method of claim 1, wherein shrinkage of the polyionic hydrogel is carried out in cell culture medium.

9. The method of claim 1, wherein the rate of shrinking can be tuned by altering one or more of: the charge density of the polyionic hydrogel or the polyionic solution, the pH of the polyionic solution, the molecular weight of the ionic polymer, or the temperature.

10. The method of claim 1, wherein the polyionic hydrogel is prepared from hydrogel macromers having a weight percentage of 5% or less in an aqueous solution.

11. A shrunken hydrogel composition, comprising water and a polyionic polymer, prepared by contacting a polyionic hydrogel with a polyionic solution including a ionic polymer having a net opposite charge from that of the polyionic hydrogel for an amount of time sufficient to decrease the volume of the polyionic hydrogel.

12. The shrunken hydrogel composition of claim 11, wherein the polyionic polymer is a hydrophilic polyionic polymer.

13. The hydrogel composition of claim 11, wherein the volume of the shrunken hydrogel composition is decreased by 5 fold.

14. The hydrogel composition of claim 11, wherein the shrunken hydrogel composition comprises one or more embedded viable cells.

15. The hydrogel composition of claim 11, wherein the hydrogel is a polyanionic hydrogel, and the polyionic solution is a polycationic solution

16. The hydrogel composition of claim 11, wherein the hydrogel is selected from the group consisting of hyaluronic acid methacrylate, gelatin methacryloyl, and alginate.

17. A method of 3D printing, comprising:
applying a composition comprising a polyionic ink in a pattern to form a structure;
crosslinking the polyionic ink to provide a polyionic hydrogel; and
contacting the polyionic hydrogel with a polyionic solution including a ionic polymer having a net opposite charge from that of the polyionic hydrogel for an amount of time sufficient to decrease the volume of the polyionic hydrogel.

18. The method of claim 17, wherein polyionic hydrogel is hydrophilic.

19. The method of claim 17, wherein the hydrogel is a polyanionic hydrogel, and the polyionic solution is a polycationic solution.

20. The method of claim 17, wherein the polyionic ink is applied by being extruded.

21. The method of claim 17, wherein the polyionic ink is applied using a stereolithographic printing apparatus.

22. The method of claim 17, wherein the polyionic ink is applied using a digital light processing printing apparatus.

23. The method of claim 17, wherein the polyionic ink is applied using vat-polymerization printing apparatus.
